(19) United States
(12) Patent Application Publication
(10) Pub. No.: US 2024/0382484 A1
(43) Pub. Date: Nov. 21, 2024

(54) COMPOSITION FOR TREATING, PREVENTING, OR AMELIORATING MELANOMA AND METHOD THEREOF

(71) Applicant: Thomas Jefferson University, Philadelphia, PA (US)

(72) Inventors: Andrew E. Aplin, Philadelphia, PA (US); Weijia Cai, Drexel Hill, PA (US)

(21) Appl. No.: 18/692,064

(22) PCT Filed: Sep. 15, 2022
(86) PCT No.: PCT/US22/76492

§ 371 Date: Mar. 14, 2024

Related U.S. Application Data
(60) Provisional application No. 63/244,291, filed on Sep. 15, 2021.

(51) Int. Cl. A61K 31/506; A61K 45/06; A61P 35/00

(52) U.S. Cl. CPC A61K 31/506; A61K 45/06; A61P 35/00

(57) ABSTRACT

Described herein is a composition for treating, preventing, and/or ameliorating melanoma in a subject in need thereof. The composition includes in certain embodiments a MEK inhibitor and a PDPK1 inhibitor. The composition includes in certain embodiments a MEK inhibitor and a Pl3K inhibitor. Further described herein is a method of treating, preventing, and/or ameliorating melanoma in a subject in need thereof. The method includes in certain embodiments administering to the subject an effective amount of a composition including a MEK inhibitor and a PDPK1 inhibitor. The method includes in certain embodiments administering to the subject an effective amount of a composition including a MEK inhibitor and a Pl3K inhibitor.

### CROSS-REFERENCE TO RELATED APPLICATIONS

The present application claims priority under 35 U.S.C. § 119(e) to U.S. Provisional Patent Application No. 63/244,291, filed Sep. 15, 2021, which is incorporated herein by reference in its entirety.

### STATEMENT REGARDING FEDERALLY SPONSORED RESEARCH

The invention was made with government support under CA160495, CA160495S1, and CA114046 awarded by the National Institutes of Health and under CA056036 under the National Cancer Institute (NCI). The government has certain rights in the invention.

### SEQUENCE LISTING

The ASCII text file named “205961-7075WO1(00287)_Seq Listing.xml” created on Sep. 5, 2022, comprising 8.90 Kbytes, is hereby incorporated by reference in its entirety.

### BACKGROUND

Melanoma is considered the most serious type of skin cancer. Melanoma accounts for about 1% of all skin cancers diagnosed in the United States, but it causes most of the deaths from skin cancer.

About 28% of melanomas patients harbor activating NRAS mutations; however, there has been little advance in targeted therapy options for NRAS mutant melanoma patients and NRAS is still considered as an “undruggable” target in melanoma.

Therefore, there is a need for new compositions and methods that treats. prevents, or ameliorates melanoma, especially melanoma that involves activating NRAS mutations. The instant application addresses that need.

### BRIEF SUMMARY

In some aspects, the present invention is directed to the following non-limiting embodiments:

### Composition

In some aspects, the present invention is directed to a composition for treating, preventing, and/or ameliorating melanoma in a subject in need thereof.

In some embodiments, the composition includes a MEK inhibitor, or a salt or solvate thereof, and a PDPK1 inhibitor, or a salt or solvate thereof.

In some embodiments, the composition includes a MEK inhibitor, or a salt or solvate thereof, and a PI3K inhibitor, or a salt or solvate thereof.

In some embodiments, the melanoma is an NRAS mutant melanoma.

In some embodiments, the subject is a mammal. In some embodiments, the subject is a human.

In some embodiments, the composition further includes a pharmaceutically acceptable excipient or carrier.

In some embodiments the MEK inhibitor comprises at least one of N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib), and N-[(2R)-2,3-dihydroxypropoxy]-3,4-difluoro-2-(2-fluoro-4-iodoanilino)benzamide (PD0325901), or a salt or solvate thereof.

In some embodiments the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or a salt or solvate thereof.

In some embodiments, the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or a salt or solvate thereof.

In some embodiments, the PI3K inhibitor is a PI3Kα inhibitor, a PI3Kβ inhibitor, a PI3Kδ inhibitor, a PI3Kα,β inhibitor, a PI3 Kβ,δ inhibitor, a PI3Kα,δ inhibitor, and/or a PI3Kα,β,δ inhibitor.

In some embodiments, the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or a salt or solvate thereof.

In some embodiments, the composition causes pyroptosis in a cell of the melanoma.

### Kit

In some aspects, the present invention is directed to a kit for treating, preventing, and/or ameliorating melanoma in a subject in need thereof.

In some embodiments, the kit includes a MEK inhibitor, or a salt or solvate thereof, and a PDPK1 inhibitor, or a salt or solvate thereof.

In some embodiments, the kit includes a MEK inhibitor, or a salt or solvate thereof, and a PI3K inhibitor, or a salt or solvate thereof.

In some embodiments, the melanoma is an NRAS mutant melanoma.

In some embodiments, the subject is a mammal. In some embodiments, the subject is a human.

In some embodiments, the kit further includes a pharmaceutically acceptable excipient or carrier. In some embodiments, the MEK inhibitor is co-formulated with the pharmaceutically acceptable excipient or carrier. In some embodiments, the PDPK1 inhibitor is co-formulated with the pharmaceutically acceptable excipient or carrier. In some embodiments, the MEK inhibitor and the PDPK1 inhibitor are each co-formulated with a same pharmaceutically acceptable excipient or carrier. In some embodiments, the MEK inhibitor and the PDPK1 inhibitor are each co-formulated with a different pharmaceutically acceptable excipient or carrier.

In some embodiments, the MEK inhibitor comprises at least one of N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib), and N-[(2R)-2,3-dihydroxypropoxy]-3,4-difluoro-2-(2-fluoro-4-iodoanilino)benzamide (PD0325901), or a salt or solvate thereof.

In some embodiments the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or a salt or solvate thereof.

In some embodiments, the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or a salt or solvate thereof.

In some embodiments, the PI3K inhibitor is a PI3Kα inhibitor, a PI3Kβ inhibitor, a PI3Kδ inhibitor, a PI3Kα,β inhibitor, a PI3Kβ,δ inhibitor, a PI3Kα,δ inhibitor, and/or a PI3Kα,β,δ inhibitor.

In some embodiments, the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or a salt or solvate thereof.

In some embodiments, the kit causes pyroptosis in a cell of the melanoma.

Method of Treating, Preventing and/or Ameliorating Melanoma

In some aspects, the present invention is directed to a method of treating, preventing, and/or ameliorating melanoma in a subject in need thereof.

In some embodiments, the method includes administering to the subject a MEK inhibitor, or a salt or solvate thereof, and a PDPK1 inhibitor, or a salt or solvate thereof.

In some embodiments, the method includes administering to the subject a MEK inhibitor, or a salt or solvate thereof, and a PI3K inhibitor, or a salt or solvate thereof.

In some embodiments, the melanoma is an NRAS mutant melanoma.

In some embodiments, the subject is a mammal. In some embodiments, the subject is a human.

In some embodiments, the MEK inhibitor comprises at least one of N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib), and N-[(2R)-2,3-dihydroxypropoxy]-3,4-difluoro-2-(2-fluoro-4-iodoanilino)benzamide (PD0325901), or a salt or solvate thereof.

In some embodiments the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or a salt or solvate thereof.

In some embodiments, the PDPK1 inhibitor includes (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or a salt or solvate thereof.

In some embodiments, the PI3K inhibitor is a PI3Kα inhibitor, a PI3Kβ inhibitor, a PI3Kδ inhibitor, a PI3Kα,β inhibitor, a PI3 Kβ,δ inhibitor, a PI3Kα,δ inhibitor, and/or a PI3Kα,β,δ inhibitor.

In some embodiments, the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or a salt or solvate thereof.

In some embodiments, the method causes pyroptosis in a cell of the melanoma.

### Method of Killing Melanoma Cell

In some aspects, the present invention is directed to a method of killing a melanoma cell.

In some embodiments, the method includes contacting the melanoma cell with a MEK inhibitor, or a salt or solvate thereof, and a PDPK1 inhibitor, or a salt or solvate thereof.

In some embodiments, the method includes contacting the cell with a MEK inhibitor, or a salt or solvate thereof, and a PI3K inhibitor, or a salt or solvate thereof.

In some embodiments, the melanoma cell has a mutation in the NRAS gene.

In some embodiments, the melanoma cell is a cultured melanoma cell. In some embodiments, the melanoma cell is a cell of a melanoma cell line. In some embodiments, the melanoma cell is a primary melanoma cell.

In some embodiments, the melanoma cell is in a subject. In some embodiments, the subject is a mammal. In some embodiments, the subject is a human.

In some embodiments, the MEK inhibitor comprises at least one of N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib), and N-[(2R)-2,3-dihydroxypropoxy]-3,4-difluoro-2-(2-fluoro-4-iodoanilino)benzamide (PD0325901), or a salt or solvate thereof.

In some embodiments the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or a salt or solvate thereof.

In some embodiments, the PDPK1 inhibitor includes (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or a salt or solvate thereof.

In some embodiments, the PI3K inhibitor is a PI3Kα inhibitor, a PI3Kβ inhibitor, a PI3Kδ inhibitor, a PI3Kα,β inhibitor, a PI3 Kβ,δ inhibitor, a PI3Kα,δ inhibitor, and/or a PI3Kα,β,δ inhibitor.

In some embodiments, the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or a salt or solvate thereof.

In some embodiments, the method causes pyroptosis in the melanoma cell.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1H: CRISPR screen identifies PDPK1, in accordance with some embodiments. FIG. 1A: Workflow of the CRISPR-Cas9 screen for genes that are essential for cell survival or act as regulators of trametinib (MEKi) sensitivity and resistance in NRAS mutant melanoma WM1361A-iCas9 #1 cells. FIGS. 1B-1D: WM1361A-iCas9 #1 cells were transduced with the Guide-it CRISPR Genome-Wide sgRNA Library. After 3 days, mCherry positive cells were determined by FACS analysis (FIG. 1). On the 24th day post infection, the cells were treated with 20 nM trametinib for further 14 days (FIG. 1C) or 0.01-0.02 nM trametinib for further 28 days (FIG. 1D). The top enriched or depleted sgRNAs targeted genes are shown. FIG. 1E, The top depleted sgRNAs targeted genes before trametinib treatment. FIG. 1F: WM1361A-iCas9 #1 cells were treated with single agents or combined inhibitors for 3 days. The growth inhibition was shown as observed inhibition (left), or was calculated as Bliss independence model (middle). The synergistic or antagonist effects (right) were calculated as Observed inhibition %−Expected inhibition %. Data are represented as the mean of three biological independent experiments. FIG. 1G: Top depleted druggable genes in low-dose trametinib treatment but not in the group before treatment (Day 24). FIG. 1H: The top hits of druggable essential genes selected with available commercial inhibitors in both WM1361A-iCas9 cells and SK-MEL-2 cells. Highlighted genes indicated the targets tested in FIG. 1F.

FIGS. 2A-2B demonstrate that PDPK1i and trametinib shows synergy, in accordance with some embodiments. FIG. 2A: NRAS mutant melanoma cell line WM1361A, WM1366, SK-MEL-30, SK-MEL-173, 1007 and 1014 were treated for 72 hours as indicated with GSK2334470 and trametinib and analyzed for cell density by IncuCyte. Synergy graphs were generated utilizing Combenefit Software with Loewe method. Data are represented as the mean from three biological independent experiments. FIG. 2B: WM1361A, WM1366, SK-MEL-30 or SK-MEL-173 cells were treated with GSK2334470 (PDPK1i) and trametinib (tram) alone or in combination (comb) at the indicated concentrations for 7 days. Cell growth was monitored by IncuCyte every 2 hours. Media were replaced twice with drugs during culture. Data are represented as the mean±SEM of three biological independent experiments. p values were calculated with an unpaired two-tailed t test. n.s., not significant.

FIGS. 3A-3J demonstrate that genetic depletion of PDPK1 sensitizes NRAS mutant cells to trametinib, in accordance with some embodiments. FIGS. 3A-3B: PDPK1 depletion by two individual sgRNAs in WM1361A (FIG. 3A) or WM1366 (FIG. 3B) cells were analyzed by immunoblot. FIGS. 3C-3D, Cell growth of WM1361A (FIG. 3C) or WM1366 (FIG. 3D) parental clones (Cas9) and PDPK1-depleted clones by two individual sgRNAs (KO1 and KO2) treated with vehicle (DMSO) or trametinib (Tram) was monitored by IncuCyte every 2 hours. Media were replaced twice with fresh drugs during culture. Data are represented as the mean±SEM of three biological independent experiments. p values were calculated with an unpaired two-tailed t test. n.s., not significant. FIGS. 3E-3F, Cellular growth in response to trametinib at the indicated doses for 5 days in WM1361A (FIG. 3E) or WM1366 (FIG. 3F) parental clone (iCas9) and PDPK1-depleted clones (KO1 and K02). Media were replaced once with drugs during culture. Data are represented as dots of three biological independent experiments. FIGS. 3G-3J, PDPK1 knockdown by ON-target control siRNAs and two individual siRNAs in SK-MEL-30 (FIG. 3G) or SK-MEL-173 cells (FIG. 3I) was analyzed by immunoblot. Cellular growth in response to trametinib at the indicated doses for 3 days in SK-MEL-30 (FIG. 3H) or SK-MEL-173 (FIG. 3J) control cells (siCtrl) and PDPK1-knockdown cells (siPDPK1s). Data are represented as dots of at least three biological independent experiments.

FIGS. 4A-4C demonstrate that combined inhibition of PDPK1 and MEK suppresses tumor growth in SK-MEL-30 xenografts, in accordance with some embodiments. FIG. 4A-4B, In Nu/J mice, SK-MEL-30 tumor growth (FIG. 4A) represented as the change in volume (mm³) over time and Kaplan-Meier survival curves (FIG. 4B) were shown from the start of treatment as indicated. Ctrl: control chow+mock injection (n=7), Tram: trametinib chow+mock injection (n=7), G4470: control chow+GSK2334470 injection (n=5), Comb: trametinib chow+GSK2334470 injection (n=7). Mice were injected and tumor volume was monitored every 3 days. Data are represented as the mean±SEM. The dotted line at 1000 mm³ marks the experimental endpoint. p values were calculated by GraphPad Prism 9.0 with Log-rank (Mantel-Cox) test. n.s., not significant. FIG. 4C: Mouse weights normalized to the initial weights at the beginning of the treatment in groups as indicated. Data are represented as the mean±SEM.

FIGS. 5A-5E demonstrate that combined inhibition of PDPK1 and MEK induces pyroptosis in NRAS mutant melanoma cells, in accordance with some embodiments. FIGS. 5A-5E: WM1361A and WM1366 cells were treated with DMSO (vehicle), PDPK1i (GSK2334470, 10M), MEKi (trametinib, 10 nM), or Comb (PDPK1i+MEKi) for 48 hours and 24 hours, respectively. Representative cell death was shown as images (FIG. 5A) and flow cytometry (FIG. 5B). Scale bar, 100 m. Annexin V⁺ PI⁺ cells were further counted as FIG. 5C. Data are represented as the mean±SEM of three biological independent experiments. p values were calculated by GraphPad Prism 9.0 with an unpaired two-tailed t test. n.s., not significant. Cell lysates (FIG. 5D) and supernatants (FIG. 5E) were analyzed by immunoblots with indicated antibodies. C or TSZ were that HaCaT immortalized human keratinocytes treated with vehicle only or with Z-VAD-FMK 20 M for 1h, then 20 ng/ml TNF-alpha+1 M Birinapant for further 24 hours.

FIGS. 6A-6E: Immune-mediated efficacy of combined inhibition of PDPK1 and MEK, in accordance with some embodiments. FIG. 6A, Tumor growth in both B6 and RAG1 KO mice, represented as the change in volume (mm³) over time, was shown from the start of treatment as indicated. Ctrl: control chow+mock injection (n=8), Tram: trametinib chow+mock injection (n=9), G4470: control chow+GSK2334470 injection (n=6), Comb: trametinib chow+GSK2334470 injection (n=9). Tumor volume was monitored on Monday, Wednesday and Friday every week. Data are represented as the mean±SEM. The dotted line at 1000 mm³ marks the experimental endpoint. X indicates a mouse sacrificed for severe ulceration on tumor. FIGS. 6B-6C, Kaplan-Meier survival curves of 1014 tumor-bearing in B6 mice only (FIG. 6B) and in B6 mice compared with RAG1 KO mice (FIG. 6C). p values were calculated by GraphPad Prism 9.0 with Log-rank (Mantel-Cox) test. n.s., not significant. FIGS. 6D-6E, 1014 tumors on C57BL/6J mice were treated with Ctrl, G4470, Tram, or Comb (n=5 for each group) for 7 days. The fold changes of tumor volume after treatment over 7 days are shown as FIG. 6D. Tumor homogenates were enriched for live immune cells using density gradient media. The CD8⁺ T cells (CD45⁺CD3⁺CD8⁺) were assessed via flow cytometry and shown as FIG. 6E. Data are represented as the mean±SD in FIG. 6D and FIG. 6E. p values were calculated by GraphPad Prism 9.0 with an unpaired two-tailed t test for FIG. 6D and a two-way ANOVA for E. n.s., not significant.

FIG. 7: Proposed model of synergistic effects of PDPK1i+MEKi on NRAS mutant melanoma, in accordance with some embodiments. A: Mutated NRAS activates both RAF-MEK-ERK and PI3K-PDPK1-AKT-mTOR signaling pathways. B: Combinatorial inhibition of PDPK1 and MEK exerts various synergistic effects, such as the cleavage of GSDME and consequent pyroptosis (C), leading to recruit CD8⁺ T cells in tumor microenvironment (D). E: Adaptive immunity-mediated response and other responses induced by PDPK1i+MEKi contribute to suppression of tumor growth. The graph was drawn with Servier Medical Art (smart dot servier dot com/).

FIGS. 8A-8E: Generation of WM1361A-iCas9 cell clone, in accordance with some embodiments. FIG. 8A: Workflow of obtaining high gene-editing efficiency clones. In brief, WM1361A cells were infected with lentivirus which express inducible cas9-P2A-GFP. After FACS sorting, individual clones were isolated and expanded. Each clone was infected by virus which express BFP and sgRNA against either GFP or BFP. Gene-editing efficiency was determined by the average of knockout efficiency of GFP or BFP. Details are available in the Materials and Methods section (Dox, doxycycline; Puro, puromycin). FIG. 8B: Three WM1361A clones were infected with virus produced by pU6-sgRNA EF1Alpha-puro-T2A-BFP (Addgene #60955, sgRNA against GFP) or pU6-sgBFP EF1Alpha-puro-T2A-BFP. After puromycin selection and DOX induction, GFP or BFP positive cells were counted by flow cytometry. FIG. 8C: Two WM1361A clones were infected with same amount of pLVXS-sgRNAs-mCherry-hyg virus. After 72 hours, mCherry positive cells were counted by flow cytometry. FIG. 8D: WM1361A clone were treated with trametinib at the indicated concentrations for 7 days. Cell growth was monitored by IncuCyte every 2 hours. Data are represented as the mean±SEM of three technical replicates. FIG. 8E: WM1361A clone were treated with trametinib at the indicated concentrations for 1h and 24 hours. Cell lysates were applied to immunoblot with indicated antibodies.

FIGS. 9A-9D: Analysis of PDPK1 and NRAS in TCGA data, in accordance with some embodiments. FIG. 9A: Correlations between PDPK1 mRNA and NRAS mRNA in TCGA human cutaneous melanoma samples. FIG. 9B: mRNA levels in NRAS mutated or NRAS wildtype cutaneous melanoma patients. FIG. 9C-9D, Survival analysis of PDPK1-high and PDPK1-low cohorts in overall human cutaneous melanoma patients (FIG. 9C) or in NRAS mutant melanoma patients (FIG. 9D).

FIGS. 10A-10B demonstrate that GSK2334470 and trametinib shows synergy in Cas9 expressed clones, in accordance with some embodiments. FIG. 10A-10B: Cas9 expressed clone WM1361A-iCas9 #1 (FIG. 10A), and WM1366-iCas9 #11 (FIG. 10B) cells were treated for 72 hours as indicated with GSK2334470 and trametinib and analyzed for cell density by IncuCyte. Synergy graphs were generated utilizing Combenefit Software with Loewe method. Data are represented as the mean from three biological independent experiments.

FIGS. 11A-11B demonstrate that PDPK1i and PD0325901 shows synergy, in accordance with some embodiments. FIG. 11A: WM1361A, WM1366, SK-MEL-30 and SK-MEL-173 cells were treated for 72 hours as indicated with GSK2334470 and PD0325901 and analyzed for cell density by IncuCyte. Synergy graphs were generated utilizing Combenefit Software with Loewe method. Data are represented as the mean from three biological independent experiments. FIG. 11B: WM1361A, WM1366, SK-MEL-30 or SK-MEL-173 cells were treated with GSK2334470 (PDPK1i) and PD0325901 (PD901) alone or in combination (Comb) at the indicated concentrations for 7 days. Cell growth was monitored by IncuCyte every 2 hours. Data are represented as the mean±SEM of three biological independent experiments. p values were calculated with an unpaired two-tailed t test. n.s., not significant.

FIGS. 12A-12F demonstrate that genetic depletion of PDPK1 sensitizes NRAS mutant cells to PD0325901, in accordance with some embodiments. FIG. 12A-12B: Cell growth of WM1361A (FIG. 12A) or WM1366 (FIG. 12B) parental clone (Cas9) and PDPK1-depleted clones (KO1 and K02) treated with vehicle (DMSO) or PD0325901 (PD) was monitored by IncuCyte every 2 hours. Media were replaced twice with drugs during culture. Data are represented as the mean±SEM of three biological independent experiment. p values were calculated with an unpaired two-tailed t test. n.s., not significant. FIGS. 12C-12D, Cellular growth in response to PD0325901 at the indicated doses for 5 days in WM1361A (FIG. 12C) or WM1366 (FIG. 12D) parental clone (iCas9) and PDPK1-depleted clones (KO1 and K02). Media were replaced once with drugs during culture. Data are represented as dots of three biological independent experiments. FIG. 12E-12F, Cellular growth in response to PD0325901 at the indicated doses for 3 days in SK-MEL-30 (FIG. 12E) or SK-MEL-173 (FIG. 12F) control cells (siCtrl) and PDPK1-knockdown cells (siPDPK1s). Data are represented as dots of at least three biological independent experiments.

FIG. 13A-13D: PDPK1 depletion delayed xenograft tumor growth in Nu/J mice, in accordance with some embodiments. FIG. 13A, Tumor growth of WM1366-iCas9 and PDPK1 knockout (KO) cells, represented as the change in volume (mm³) over time, was shown from the start of injection to the start of treatment as indicated. Cyan dots indicated sacrifice of mice due to lack of visible tumors. The dotted line at 50 mm³ indicates the size at which trametinib treatment was started. FIG. 13B: Time for tumor growth of WM1366-iCas9 cells to the start of treatment in male and female mice. Lines represent median number of days from the time of tumor implantation to the treatment size of 50 mm³. The mouse without tumor formation was monitored for 105 days. p values were calculated with an unpaired two-tailed t test. FIG. 13C: Tumor growth of WM1366-iCas9 cells, represented as the change in volume (mm³) over time, was shown from the start of treatment as indicated. Ctrl: control chow, Tram: trametinib chow. The dotted line at 1000 mm³ marks the experimental endpoint. FIG. 13D: Kaplan-Meier survival curves of WM1366-iCas9 tumor-bearing in female or male mice as indicated. p values were calculated by GraphPad Prism 9.0 with Log-rank (Mantel-Cox) test. n.s., not significant.

FIGS. 14A-14B: Sex disparities in SK-MEL-30 xenografts, in accordance with some embodiments. FIG. 14A-14B: In Nu/J mice, individual tumor growth (FIG. 14A) represented as the change in volume (mm³) over time and Kaplan-Meier survival curves (FIG. 14B) were shown from the start of treatment as indicated. Ctrl: control chow+mock injection, Tram: trametinib chow+mock injection, G4470: control chow+GSK2334470 injection, Comb: trametinib chow+GSK2334470 injection. Mice were injected and tumor volume was monitored every 3 days. Data are represented as the mean±SEM. The dotted line at 1000 mm³ marks the experimental endpoint. p values were calculated by GraphPad Prism 9.0 with Log-rank (Mantel-Cox) test. n.s., not significant.

FIGS. 15A-15C demonstrate that combined inhibition of PDPK1 and MEK inhibits p-S6. FIG. 15A: Reverse-phase protein array analysis of WM1361A and WM1366 cells. The cells were treated with vehicle (DMSO), either pharmacologic inhibition or genetic depletion of PDPK1 only, MEK inhibitor only and the combinations as indicated. All samples were harvested in three biological independent experiments. Proteins levels were normalized to the mean. Antibodies were limited to those that were differentially expressed (BHFDR <0.05) in at least three of the four comparisons between combination and control samples. FIG. 15B: WM1361A and WM1366 cells were treated with PD0325901 and GSK2334470 as indicated for 24 hours. Cell lysates were applied to immunoblot with indicated antibodies. FIG. 15C: WM1361A and WM1366 parental clones (iCas9) and PDPK1-depleted clones by two individual sgRNAs (KO1 and K02) treated with trametinib as indicated for 24 hours. Cell lysates were applied to immunoblot with indicated antibodies.

FIGS. 16A-16B: Combined inhibition of PI3K, AKT, or mTOR with MEK in NRAS mutant melanoma cells, in accordance with some embodiments. FIG. 16A: WM1361A and WM1366 cells were treated for 72 hours with indicated inhibitors and analyzed for cell density by IncuCyte. Synergy graphs were generated utilizing Combenefit Software with Loewe method. Data are represented as the mean from three biological independent experiments. FIG. 16B: WM1361A and WM1366 cells were treated with indicated inhibitors for 24 hours. Cell lysates were applied to immunoblot with indicated antibodies.

FIGS. 17A-17D: 1014 allograft model in B6 and RAG1 KO mice, in accordance with some embodiments. FIG. 17A-17B: 1014 cells were treated with GSK2334470 (PDPK1i) and trametinib (Tram) alone (FIG. 17A) or PD0325901 (PD901) alone (FIG. 17B) or in combination (comb) at the indicated concentrations for 7 days. Cell growth was monitored by IncuCyte every 2 hours. Data are represented as the mean±SEM of three biological independent experiments. p values were calculated using a fitted model as described in Materials and Methods between indicated groups. n.s., not significant. FIG. 17C, Tumor volume of 1014 isografts treated in B6 mice. Ctrl: control chow+mock injection, Tram: trametinib chow+mock injection, G4470: control chow+GSK2334470 injection, Comb: trametinib chow+GSK2334470 injection. The dotted line at 1000 mm³ marks the experimental endpoint. Data are represented as the mean±SEM. FIG. 17D, Mouse weight (gram) from enrollment time in groups as indicated in B6 and RAG1 KO mice. Data are represented as the mean±SEM.

FIGS. 18A-18D: Representative gating strategy for flow cytometry, in accordance with some embodiments. Representative FACS plots showing the gating strategy for immune cells taken from 1014 tumors on day 7 from C57BL/6J mice treated with Ctrl: control chow+mock injection, Tram: trametinib chow+mock injection, GSK: control chow+GSK2334470 injection, Comb: trametinib chow+GSK2334470 injection (n=5 for each group). Tumor homogenate was enriched for live immune cells using density gradient media. FIG. 18A: Plots identify single cells positive for CD45. FIG. 18B: The CD45⁺ population was assessed for CD8⁺ T cells (CD3⁺CD8⁺), CD4⁺ T cells (CD3⁺CD8⁺), B cells (CD19⁺), and NK cells (NK1.1⁺). FIG. 18C: Dendritic cells were identified from the CD45⁺ population in (FIG. 18A) by gating on CD11b⁺CD11C⁺ cells that were negative for F4/80. FIG. 18D: Macrophages were identified from the CD45⁺ population in (FIG. 18A) by gating on the CD11b⁺F4/80⁺ population and were further assessed for the presence of CD11c (M1-like macrophages) and GR-1 (MDSC-like macrophages).

FIGS. 19A-19B: Immune cells taken from 1014 tumors on day 7 from C57BL/6J mice treated with Ctrl: control chow+mock injection, Tram: trametinib chow+mock injection, G4470: control chow+GSK2334470 injection, Comb: trametinib chow+GSK2334470 injection (n=5 for each group). Tumor homogenate was enriched for live immune cells using density gradient media. The immune cell subpopulations were assessed via flow cytometry, determined as shown in FIGS. 17A-17D, analyzed with Flowjo and presented as indicated. Data are represented as the mean±SD. p values were calculated by GraphPad Prism 9.0 with an unpaired two-tailed t test. No significant difference was found between Comb and Ctrl.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Mutated RAS family members are oncogenic drivers in more than 30% of human cancers (Fernandez-Medarde et al. Genes Cancer 2, 344-358, 2011). Alternative approaches to target RAS include interfering with the post-translational modification of RAS and inhibition of downstream RAS effectors, but have not been proven effective clinically.

New treatment strategies for RAS mutant tumors are particularly relevant in cutaneous melanoma in which ˜28% of patients harbor activating NRAS mutations (Cell. 161(7):1681-96. doi: 10.1016/j.cell.2015.05.044, 2015). Targeted therapy and immunotherapy have significantly improved overall survival for patients with BRAF mutations. If NRAS mutant melanomas are non-responsive to immunotherapy, patients have no FDA-approved targeted therapies, a poor prognosis, and bleak disease outcomes (Munoz-Couselo et al., Onco Targets Ther 10, 3941-3947, doi:10.2147/OTT.S117121, 2017). MEK inhibitors (MEKi), which target the hyper-activated MEK-ERK1/2 pathway downstream of mutant NRAS, were among the most promising candidates. Unfortunately, Phase I to II trials of MEKi in NRAS mutant melanomas have shown limited clinical efficacy and invariably lead to resistance after only a few months. The mechanisms of resistance remain poorly understood. Binimetinib, the first MEKi to be evaluated in a Phase III clinical trial for NRAS mutant melanoma patients, showed modest advantage in progression-free survival over standard chemotherapy (dacarbazine), but the survival benefit was deemed insufficient to support FDA approval of binimetinib (Dummer et al. Lancet Oncol 18, 435-445, doi:10.1016/S1470-2045(17)30180-8, 2017).

The present study utilized a genome-wide CRISPR/Cas9-based screen to identify PDPK1 as a therapeutic target to enhance the efficacy of MEKi in NRAS mutant melanoma cells. Genetic or pharmacological depletion of PDPK1 showed synergy with MEKi with regards to their inhibition of cell growth in NRAS mutant melanoma cell lines. The synergistic effects of PDPK1 loss and MEKi were validated in various NRAS mutant melanoma cell lines via pharmacological and molecular approaches. Furthermore, combined PDPK1 inhibitor (also referred to as “PDPK1i” herein) and MEK inhibitor (also referred to as “MEKi” herein) treatment induced gasdermin E-associated pyroptosis. In an immune competent allograft model, PDPK1i+MEKi increased the ratio of intratumoral CD8⁺ T cells, delayed tumor growth and prolonged survival whereas PDPK1i+MEKi showed a significantly weaker potency an isogenic immune deficient model.

The discovery described herein is unexpected. NRAS mutations activate both MAPK pathway and PI3K-AKT-mTOR pathway. MEK is involved in the MAPK pathway, and PDPK1 and AKT are both involved in the PI3K-AKT-mTOR pathway. It has been reported that dual MEK/AKT inhibition did not yield clinical benefit in metastatic NRAS mutant melanoma (Algazi et al. Pigment Cell Melanoma Res 31, 110-114, doi:10.1111/pcmr.12644, 2018), and that report is confirmed by the present study. Indeed, the present study found that pan AKT inhibitor (AKTi) (MK-2206 2HCl) and mTORC1/2 inhibitor (mTORC1/2i) (AZD8055) showed reduced synergy and even antagonistic effects with MEK inhibitor. Therefore, it was expected that the combination of a MEK inhibitor and a PDPK1 inhibitor would have not been able to achieve such a profound delayed of NRAS mutant tumor growth.

Accordingly, the instant specification is directed to the following aspects:

In some aspects, the instant specification is directed to a composition for treating, preventing, and/or ameliorating melanoma.

In some aspects, the instant specification is directed to a method of treating, preventing, and/or ameliorating melanoma.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

Composition or Kit for Treating, Preventing, and/or Ameliorating Melanoma

In some embodiments, the instant specification is directed to a composition for treating or ameliorating melanoma in a subject in need thereof, including a MEK inhibitor and a PDPK1 inhibitor. In some embodiments, the method composition includes a MEK inhibitor, or a salt or solvate thereof, and a PI3K inhibitor, or a salt or solvate thereof.

In some embodiments, the instant specification is directed to a kit for treating or ameliorating melanoma in a subject in need thereof, including a MEK inhibitor, or a salt or solvate thereof, and a PDPK1 inhibitor, or a salt or solvate thereof. In some embodiments, the kit includes a MEK inhibitor, or a salt or solvate thereof, and a PI3K inhibitor, or a salt or solvate thereof.

In some embodiments, in the kit, the MEK inhibitor, or a salt or solvate thereof, and the PDPK1 inhibitor, or a salt or solvate thereof, (or the MEK inhibitor, or a salt or solvate thereof, and the PI3K inhibitor, or a salt or solvate thereof) are not mixed with each other, and are suitable for being administered separately, such as via different routes of administration.

In some embodiments, the melanoma is an NRAS mutant melanoma.

In some embodiments, the subject is a mammal. In some embodiments, the subject is a human.

In some embodiments, the composition or the kit causes pyroptosis in a cell of the melanoma.

In certain embodiments, the MEK inhibitor is any compound disclosed in International Application No. PCT/JP2005/011082, having an International filing date of Jun. 10, 2005; International Publication Number WO 2005/121 142 and International Publication date of Dec. 22, 2005, the entire disclosures of which are hereby incorporated by reference. In certain embodiments, the MEK inhibitor is

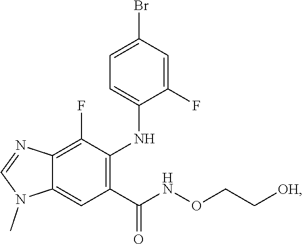 [SMILES: Cn1cnc2c(F)c(Nc3ccc(Br)cc3F)c(C(=O)NOCCO)cc21]

5-((4-bromo-2-fluorophenyl)amino)-4-fluoro-N-(2-hydroxyethoxy)-1-methyl-1H-benzo[d]imidazole-6-carboxamide (binimetinib). In certain embodiments, the MEK inhibitor is

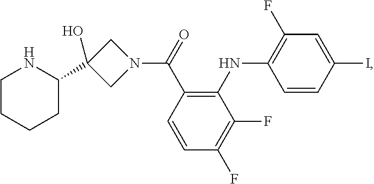 [SMILES: O=C(c1ccc(F)c(F)c1Nc1ccc(I)cc1F)N1CC(O)([C@@H]2CCCCN2)C1]

(S)-(3,4-difluoro-2-((2-fluoro-4-iodophenyl)amino)phenyl)(3-hydroxy-3-(piperidin-2-yl)azetidin-1-yl)methanone (cobimetinib). In certain embodiments, the MK inhibitor is

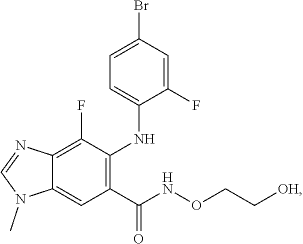 [SMILES: Cn1cnc2c(F)c(Nc3ccc(Br)cc3F)c(C(=O)NOCCO)cc21]

5-((4-bromo-2-chlorophenyl)amino)-4-fluoro-N-(2-hydroxyethoxy)-1-methyl-1H-benzo[d]imidazole-6-carboxamide (selumetinib). In certain embodiments, the MEK inhibitor is

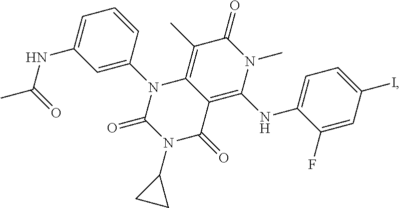 [SMILES: CC(=O)Nc1cccc(-n2c(=O)n(C3CC3)c(=O)c3c(Nc4ccc(I)cc4F)n(C)c(=O)c(C)c32)c1]

N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib). In certain embodiments, the MEK inhibitor is

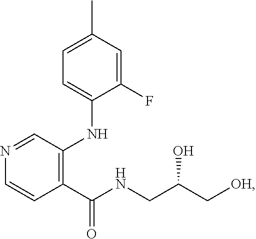 [SMILES: Cc1ccc(Nc2cnccc2C(=O)NC[C@H](O)CO)c(F)c1]

(S)—N-(2,3-dihydroxypropyl)-3-((2-fluoro-4-iodophenyl)amino)isonicotinamide (pimasertib). In certain embodiments, the MEK inhibitor is

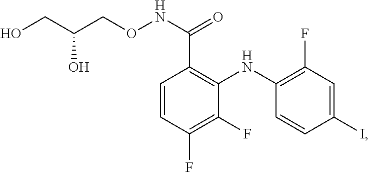 [SMILES: O=C(NOC[C@H](O)CO)c1ccc(F)c(F)c1Nc1ccc(I)cc1F]

(R)—N-(2,3-dihydroxypropoxy)-3,4-difluoro-2-((2-fluoro-4-iodophenyl)amino)benzamide (PD-0325901). In certain embodiments, the MEK inhibitor is

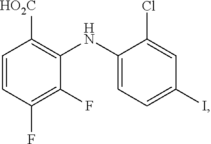 [SMILES: O=C(O)c1ccc(F)c(F)c1Nc1ccc(I)cc1Cl]

2-((2-chloro-4-iodophenyl)amino)-3,4-difluorobenzoic acid (ATR-002).

In certain embodiments, the MEK inhibitor is

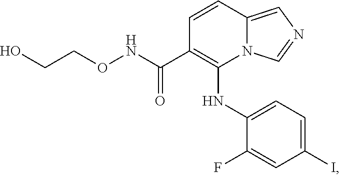 [SMILES: O=C(NOCCO)c1ccc2cncn2c1Nc1ccc(I)cc1F]

5-((2-fluoro-4-iodophenyl)amino)-N-(2-hydroxyethoxy)imidazo[1,5-a]pyridine-6-carboxamide (GDC-0623). In certain embodiments, the MEK inhibitor is

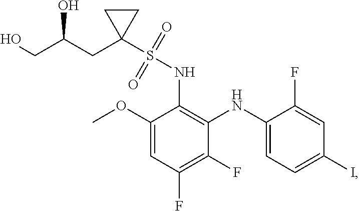 [SMILES: COc1cc(F)c(F)c(Nc2ccc(I)cc2F)c1NS(=O)(=O)C1(C[C@H](O)CO)CC1]

(S)—N-(3,4-difluoro-2-((2-fluoro-4-iodophenyl)amino)-6-methoxyphenyl)-1-(2,3-dihydroxypropyl)cyclopropane-1-sulfonamide (refametinib). In certain embodiments, the MEK inhibitor is

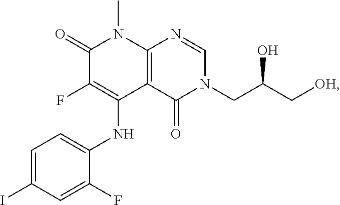 [SMILES: Cn1c(=O)c(F)c(Nc2ccc(I)cc2F)c2c(=O)n(C[C@@H](O)CO)cnc21]

(R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733). In certain embodiments, the MEK inhibitor is REC4881.

In some embodiments, the MEK inhibitor comprises N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib) or N-[(2R)-2,3-dihydroxypropoxy]-3,4-difluoro-2-(2-fluoro-4-iodoanilino)benzamide (PD0325901).

Non-limiting examples of PDPK1 inhibitors include GSK2334470 ((3S,6R)-1-[6-(3-Amino-1H-indazol-6-yl)-2-(methylamino)-4-pyrimidinyl]-N-cyclohexyl-6-methyl-3-piperidinecarboxamide),

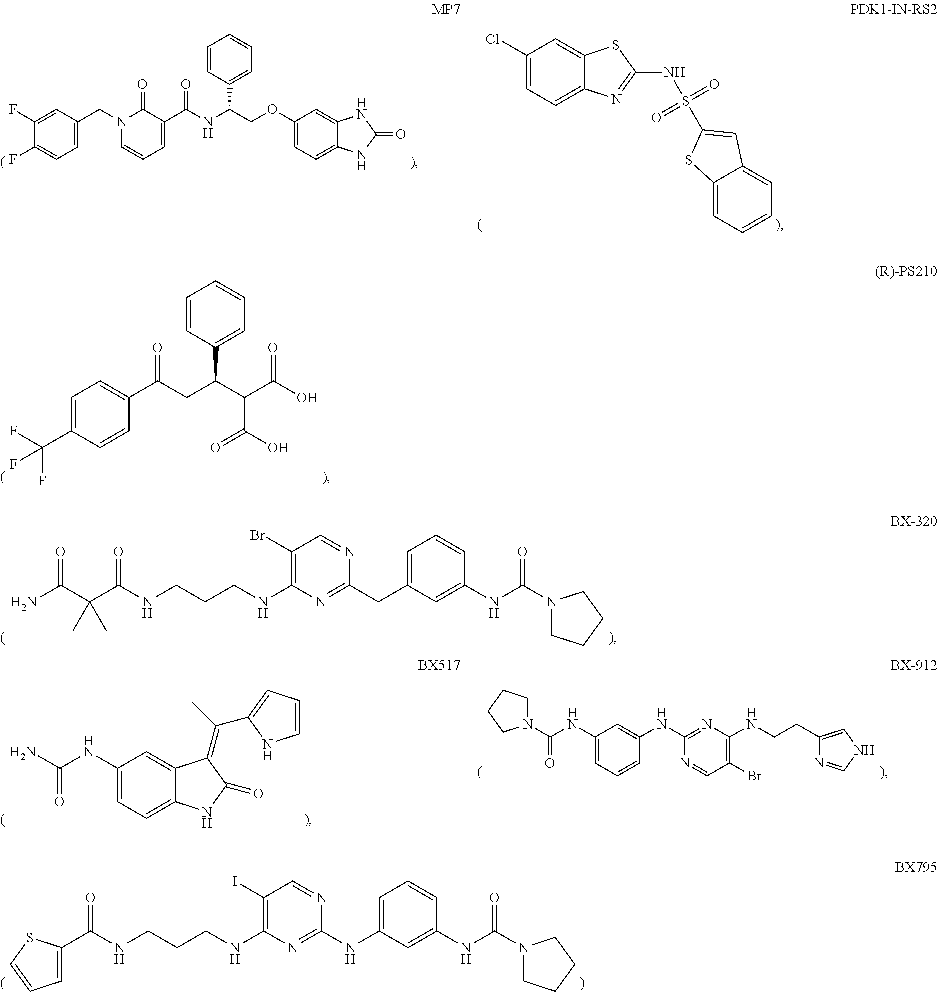

and the like.

In some embodiments, the PDPK1 inhibitor is a compound as described in WO2010059658A1 and WO2010019637A1 (the entireties of these documents are hereby incorporated herein by reference).

In some embodiments, the PDPK1 inhibitor is a compound as described in Medina (J. Med. Chem. 2013, 56, 7, 2726-2737) or Sestito et al. (Expert Opinion on Therapeutic Patents Volume 29, Issue 4, Pages 271-282, 2019).

In some embodiments, the PDPK1 inhibitor is (3S,6R)-1-[6-(3-Amino-1H-indazol-6-yl)-2-(methylamino)-4-pyrimidinyl]-N-cyclohexyl-6-methyl-3-piperidinecarboxamide (also referred to as GSK2334470).

In some embodiments, the PI3K inhibitor is a PI3Kα inhibitor, a PI3Kβ inhibitor, a PI3Kδ inhibitor, a PI3Kα,β inhibitor, a PI3 Kβ,δ inhibitor, a PI3Kα,δ inhibitor, and/or a PI3Kα,β,δ inhibitor. PI3K inhibitors are described in, for example, Vanhaesebroeck et al. (Nature Reviews Drug Discovery volume 20, pages 741-769 (2021)), the entirety of the reference is hereby incorporated herein by reference.

Non-limiting examples of PI3K inhibitors include acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, ZSTK474, and the like, or a salt or solvate thereof.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and 35S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts.

Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

In some embodiments, the composition or the kit for treating, preventing or ameliorating melanoma further includes a pharmaceutically acceptable excipient or carrier.

Method of Treating, Preventing, and/or Ameliorating Melanoma

In some embodiments, the instant specification is directed to a method of treating, preventing or ameliorating melanoma, including administering to a subject in need thereof an effective amount of a MEK inhibitor, or a salt or solvate thereof, and a PDPK1 inhibitor, or a salt or solvate thereof. In some embodiments, the method includes administering to the subject in need thereof an effective amount of a MEK inhibitor, or a salt or solvate thereof, and a PI3K inhibitor, or a salt or solvate thereof.

In some embodiments, the MEK inhibitor, or a salt or solvate thereof, and the PDPK1 inhibitor, or a salt or solvate thereof, (or the MEK inhibitor, or a salt or solvate thereof, and the PI3K inhibitor, or a salt or solvate thereof) are administered as a composition.

In some embodiments, the MEK inhibitor, or a salt or solvate thereof, and the PDPK1 inhibitor, or a salt or solvate thereof, (or the MEK inhibitor, or a salt or solvate thereof, and the PI3K inhibitor, or a salt or solvate thereof) are administered separately. In some embodiments, the MEK inhibitor, or a salt or solvate thereof, and the PDPK1 inhibitor, or a salt or solvate thereof, (or the MEK inhibitor, or a salt or solvate thereof, and the PI3K inhibitor, or a salt or solvate thereof) are administered via the same route of administration. In some embodiments, the MEK inhibitor, or a salt or solvate thereof, and the PDPK1 inhibitor, or a salt or solvate thereof, (or the MEK inhibitor, or a salt or solvate thereof, and the PI3K inhibitor, or a salt or solvate thereof) are administered via different routes of administration. Non-limiting examples of routes of administration are described elsewhere herein, such as in the “Administration/Dosage/Formulations” section.

In some embodiments, the melanoma is an NRAS mutant melanoma.

In some embodiments, the subject is a mammal. In some embodiments, the subject is a human.

In some embodiments, the method causes pyroptosis in a cell of the melanoma.

In some embodiments, the MEK inhibitor, the PDPK1 inhibitor, or the PI3K inhibitor is the same as or similar to those as described in the “Composition and/or Kit for Treating or Ameliorating Melanoma” section herein.

### Method of Killing Melanoma Cell

In some embodiments, the present invention is directed to a method of killing a melanoma cell.

In some embodiments, the method includes contacting the melanoma cell with a MEK inhibitor, or a salt or solvate thereof, and a PDPK1 inhibitor, or a salt or solvate thereof. In some embodiments, the method includes contacting the melanoma cell with a MEK inhibitor, or a salt or solvate thereof, and a PI3K inhibitor, or a salt or solvate thereof.

In some embodiments, the melanoma cell has a mutation in the NRAS gene.

In some embodiments, the melanoma cell is a cultured melanoma cell. In some embodiments, the melanoma cell is a cell of a melanoma cell line. In some embodiments, the melanoma cell is a primary melanoma cell.

In some embodiments, the melanoma cell is in a subject. In some embodiments, the subject is a mammal. In some embodiments, the subject is a human.

In some embodiments, the method causes pyroptosis in the melanoma cell.

In some embodiments, the MEK inhibitor, the PDPK1 or the PI3K inhibitor, or any salt or solvate, thereof, is the same as or similar to those as described in the “Composition and/or Kit for Treating or Ameliorating Melanoma” section herein.

### Combination Therapies

In some embodiments, the method of treating, ameliorating, and/or preventing the neurodegenerative condition or the method of reversing or preventing formation and/or enlargement of axonal spheroids includes administering to the subject the effective amount of at least one compound and/or composition contemplated within the disclosure.

In some embodiments, the composition for treating neurodegenerative condition includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating neurodegenerative conditions, and/or at least one additional agent that treats one or more diseases or disorders contemplated herein.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 g to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of neurodegenerative conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the neurodegenerative condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Genome-Wide CRISPR/Cas9-Based Screen Identifies Potential Synergistic Targets of MEKi

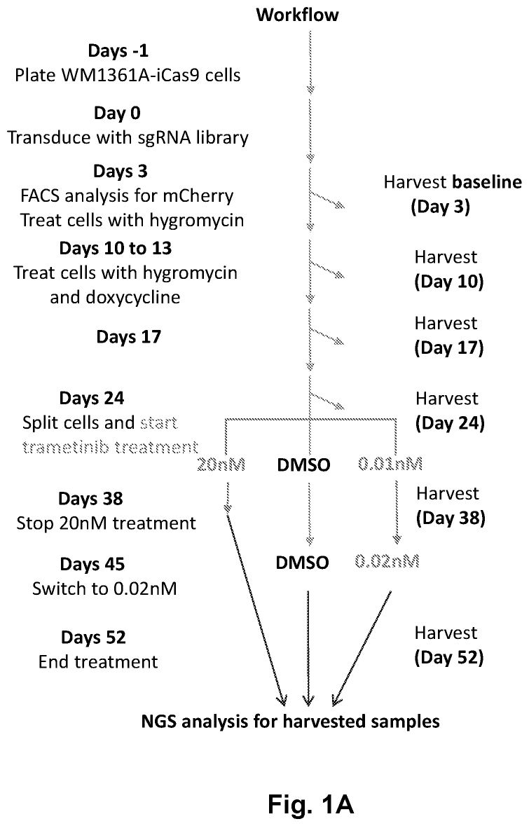

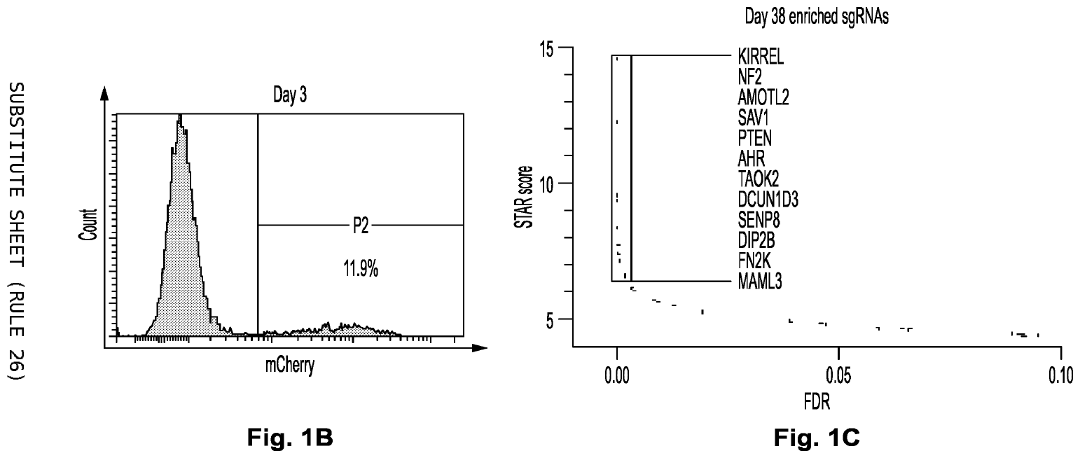

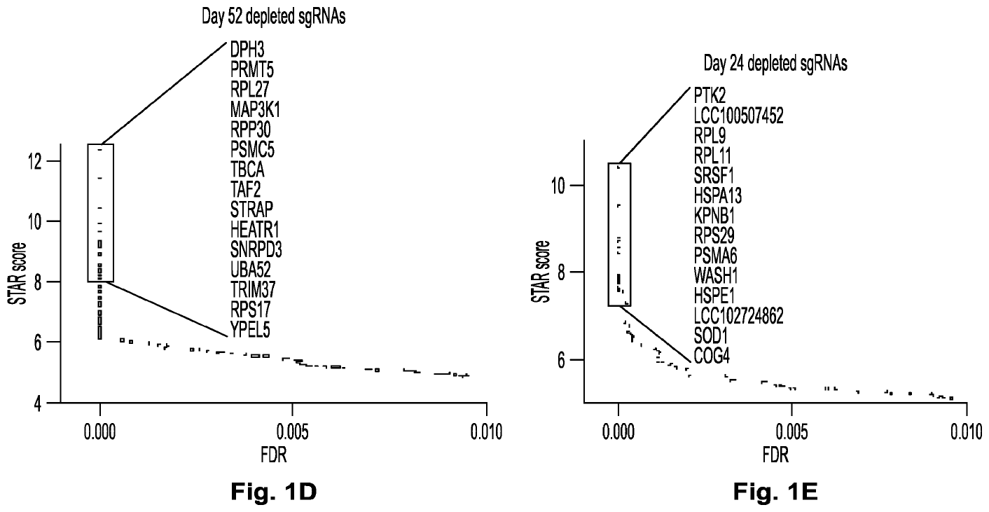

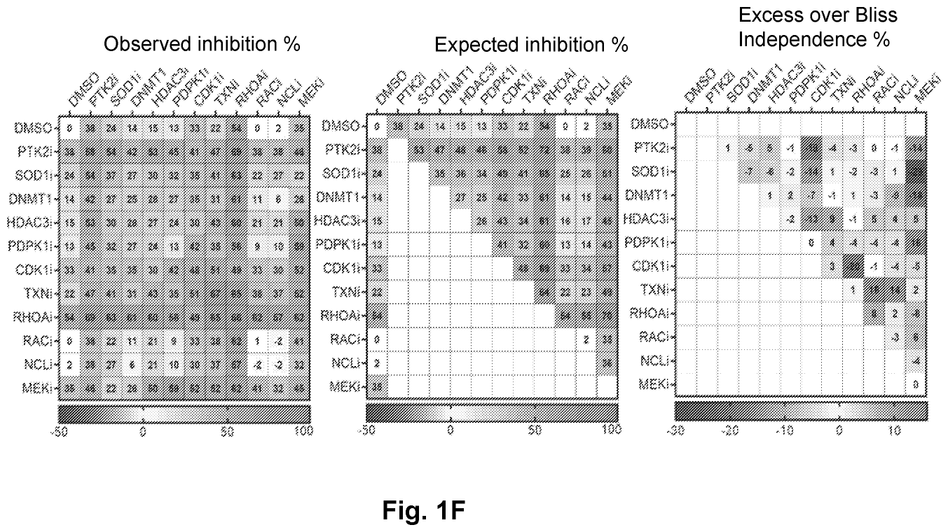

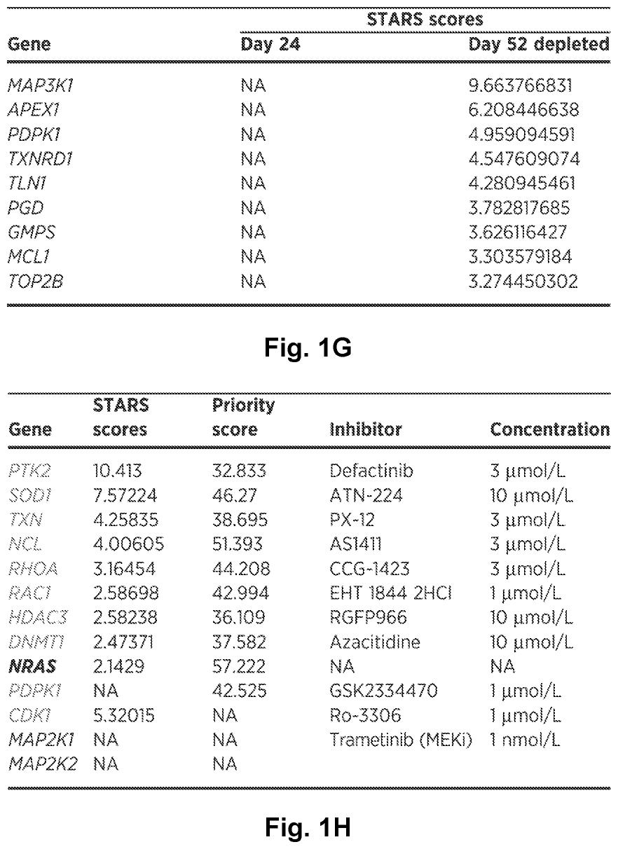

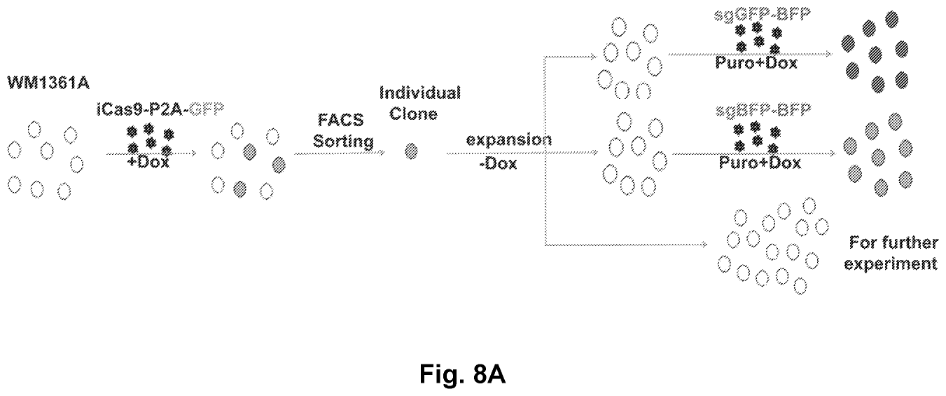

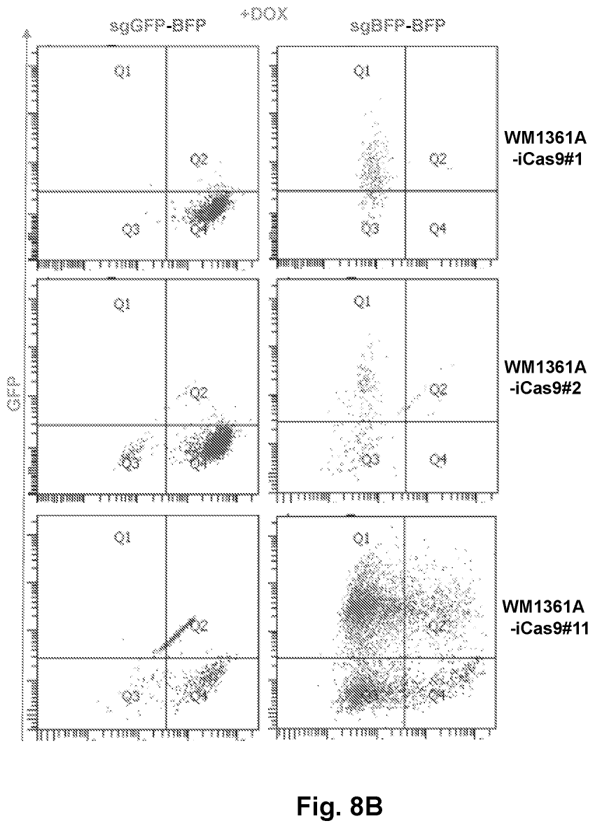

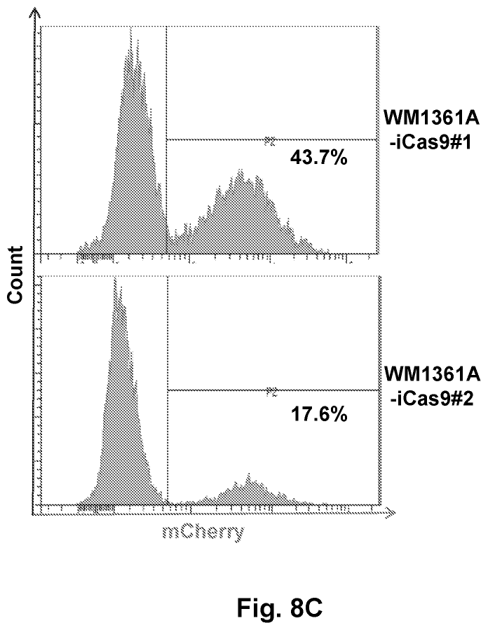

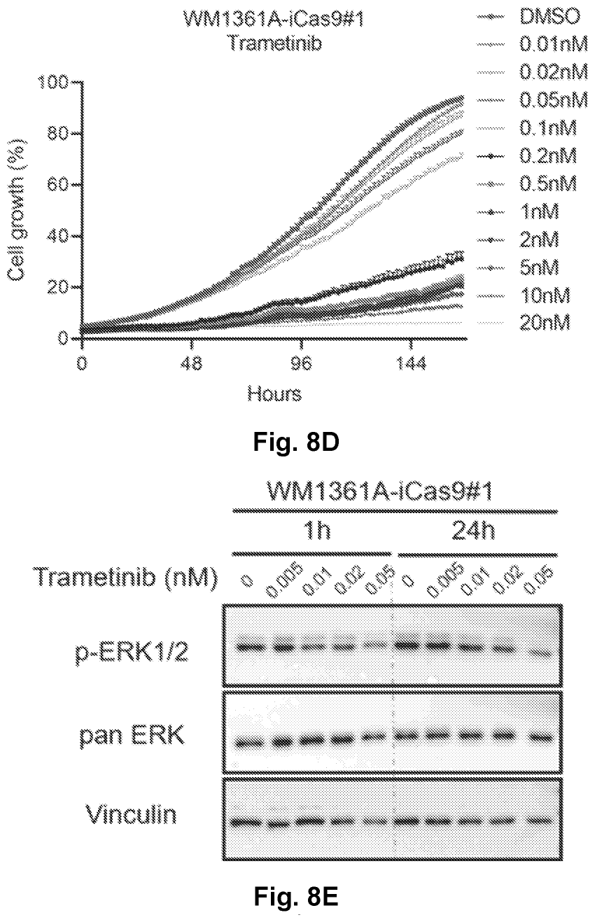

To establish the platform for a CRISPR-Cas9-based screen, Cas9 protein was expressed in the NRAS mutant melanoma cell line, WM1361A. Cas9 expression level was induced by doxycycline and monitored by GFP linked to Cas9 via a self-cleavage peptide P2A (FIG. 8A). The present study utilized a lentiviral vector expressing BFP and sgRNAs against either GFP or BFP to check the gene-editing efficiency of different clones (FIG. 8A-8B). The present study utilized a lentivirus expressing mCherry to identify one clone (WM1361A-iCas9 #1) with high transduction efficiency to facilitate further large-scale infection (FIG. 8C). WM1361A-iCas9 #1 cells were treated with various doses of trametinib (also known as GSK1120212 or Mekinist) to test for proliferation under MEKi treatment (FIG. 8D-8E). Approximately 2.34×10⁸ WM1361A-iCas9 #1 cells were infected with Takara Bio Guide-It™ CRISPR Genome-wide sgRNA library (76,610 sgRNAs targeting 19,114 genes) with an infection efficiency of 11.9% (FIGS. 1A-1B). After 28 days, a three-armed screen was performed in WM1361A-iCas9 #1 cells treated with DMSO vehicle control, low doses (0.01 nmol/L to 0.02 nmol/L) and a high dose (20 nmol/L, 20 nM) of trametinib (FIG. 1A).

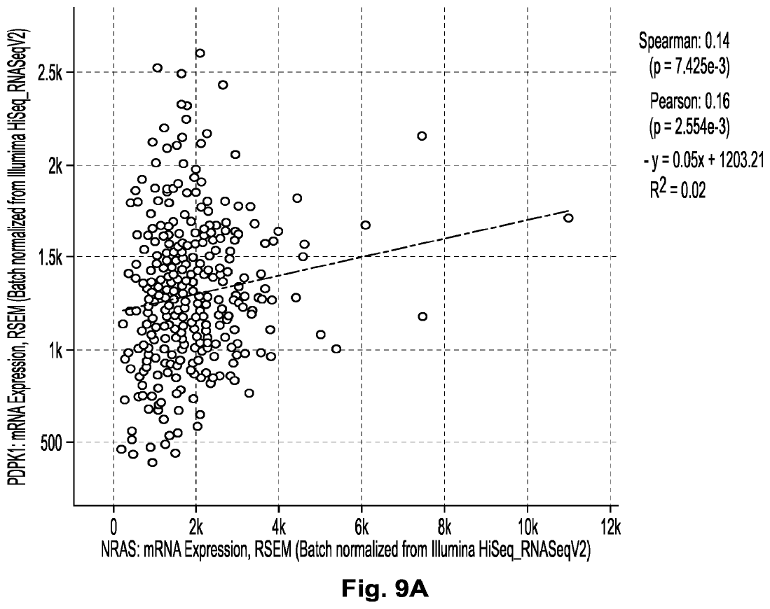

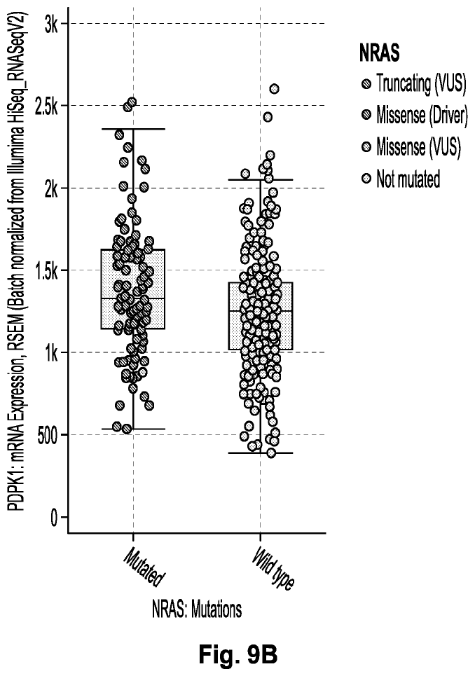

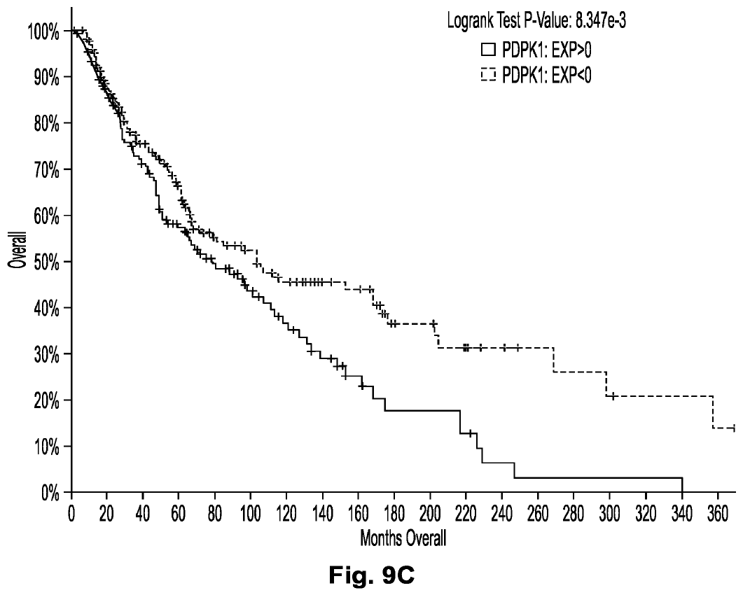

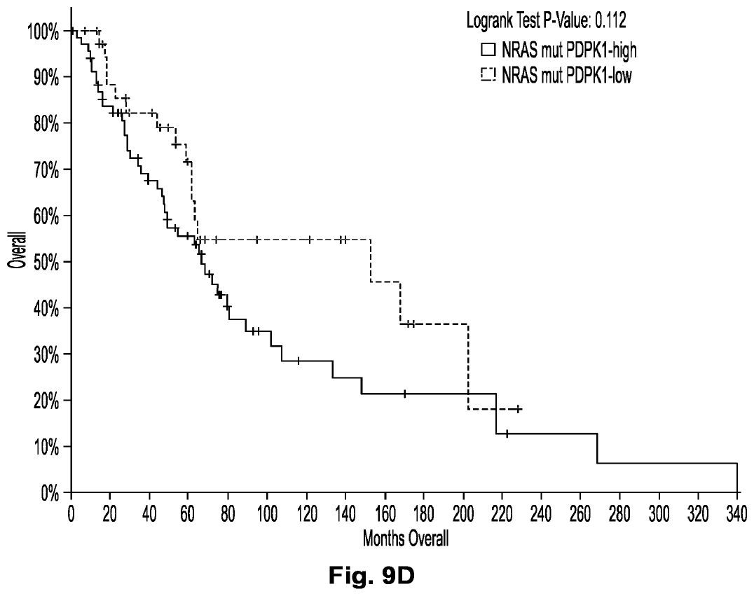

Using a gene-ranking algorithm for genetic perturbation screens, the STARS method, a ranking algorithm designed to identify enriched genes in genetic knockdown and knockout screens, the screening results for the high dose trametinib arm revealed enrichment of tumor suppressor genes such as NF2 and PTEN (FIG. 1C), suggesting that loss of these genes contributes to trametinib resistance. A top hit of the list of genes targeted by sgRNAs that are depleted from cells treated with low dose of trametinib (FIG. 1D) was MAP3KL. After exclusion of genes targeted by sgRNAs that were depleted from cells before trametinib treatment (FIG. 1E) and cross-referencing the results with Therapeutic Targets Database (TTD) v7.1.01, the present study obtained a list of genes which can be pharmaceutically targeted and potentially promotes trametinib efficacy (FIG. 1G). The present study further studied PDPK1, which encodes Phosphoinositide-dependent kinase-1 (PDPK1 or PDK1). PDPK1 mRNA expression level showed a modest positive correlation (Pearson=0.16) with NRAS mRNA expression level (FIG. 9A). PDPK1 mRNA expression level was higher in NRAS mutant melanoma patients than in NRAS wild-type patients (FIG. 9B). Lower PDPK1 expression level predicted a better prognosis in melanoma patients (p=0.008) (FIG. 9C). In NRAS mutant patients, there was a similar tendency even though it did not reach statistical significance (p=0.112) (FIG. 9D). These data hint that PDPK1 may play a tumor-promoting role in NRAS mutant melanoma patients.

The present study further analyzed druggable essential genes with high STARS scores (>2.14 of NRAS) in WM1361A-iCas9 cells which were also identified as therapeutic targets with high priority scores (>30) in NRAS mutant melanoma SK-MEL-2 cells in a published database. The top hits included PTK2, SOD1, TXN and NCL (FIG. 1F). Since potential synergistic combinations of MEKi and essential genes could not be detected in the negative screen, the growth response of WM1361A-iCas9 cells to a panel of available specific inhibitors to druggable essential genes was assessed (FIG. 1H). In this panel, PDPK1i and MEKi were also added as a validation of synergy. Most combinations showed modest synergy or antagonistic effects as calculated by the Bliss independence model whereas PDPK1i (GSK2334470) was synergistic with MEKi (trametinib) (FIG. 1F).

### Example 2: Pharmacological Validation of PDPK1 as a Target to Enhance MEKi Efficacy in NRAS Mutant Melanoma

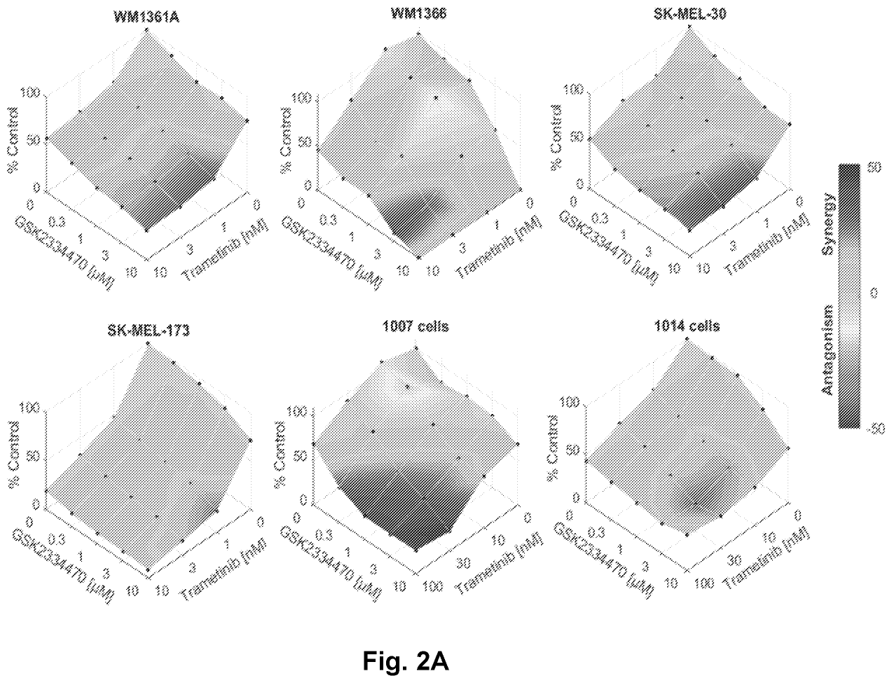

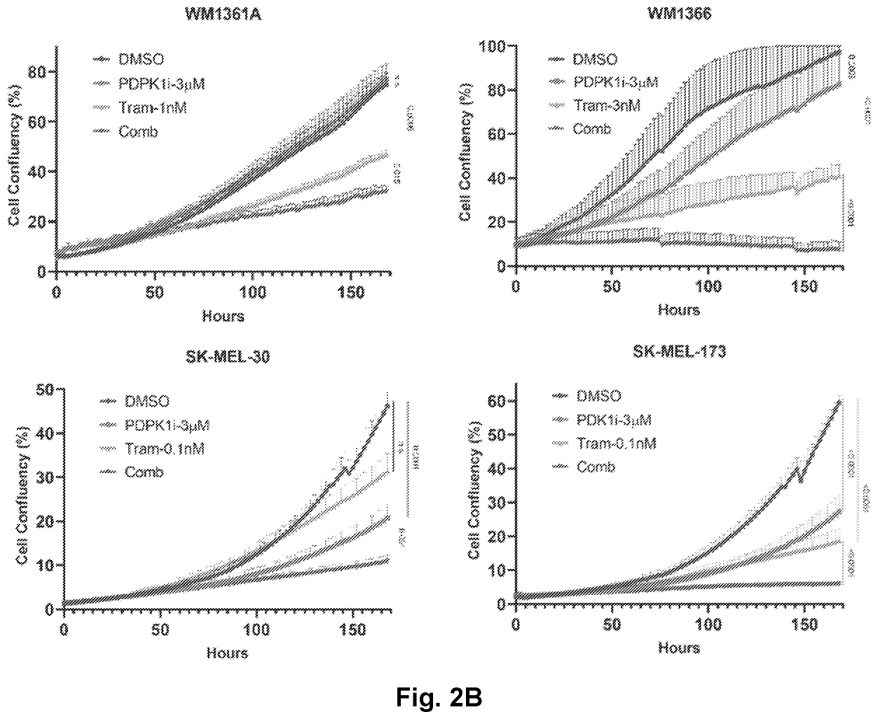

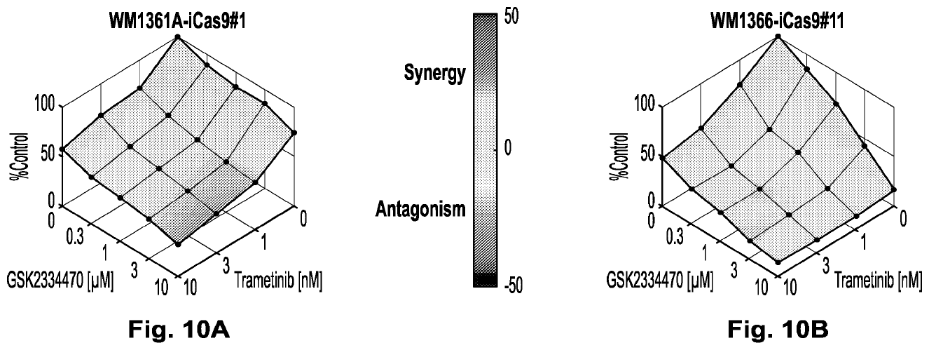

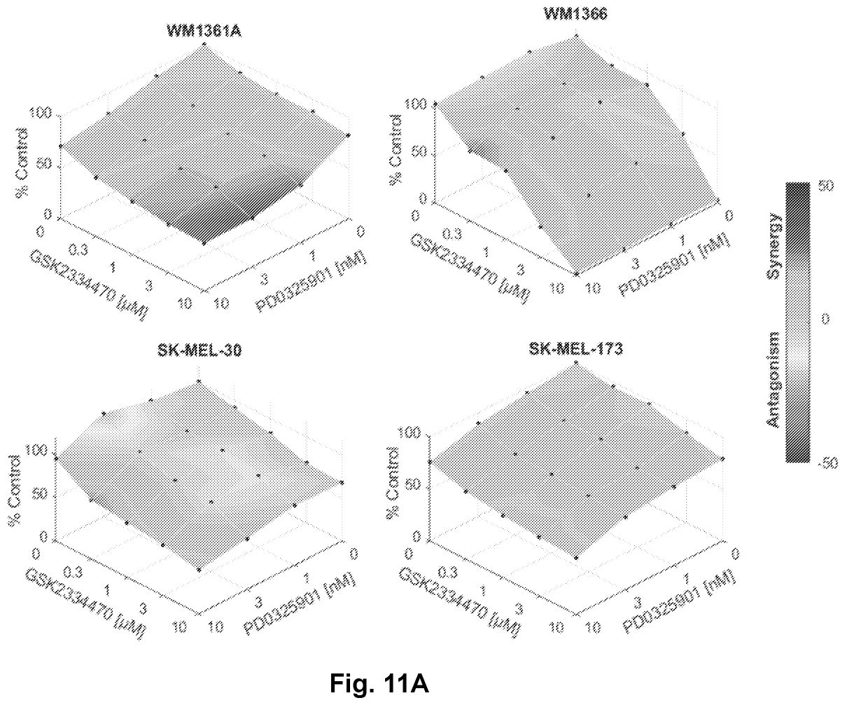

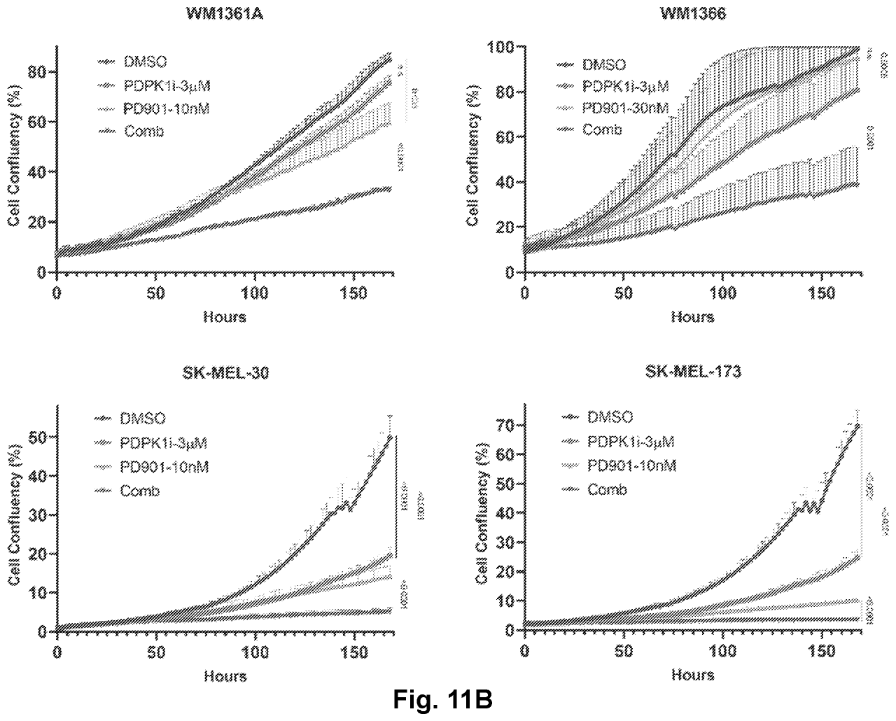

To further analyze synergistic effects, the growth response of a panel of several NRAS mutant cells to titrated PDPK1i (GSK2334470) and MEKi (trametinib) combinations was assessed. Synergistic inhibition of cell viability was observed in a wide range of drug concentrations in two Cas9-expressed cell lines (FIGS. 10A-10B), four parental human NRAS mutant melanoma cell lines and two mouse Nras mutant melanoma cell lines (FIG. 2A) after 3-day drug treatments. To exclude the possibility that the synergy is confined to trametinib, the present study tested the synergistic inhibition of PDPK1i with another MEKi, PD0325901 (PD901) which targets MEK via a different mechanism from trametinib. The present study obtained similar results, which indicated broad synergistic effects of PDPK1 and MEK inhibition (FIG. 11A). In addition, combining PDPK1i with trametinib or PD901 profoundly inhibited cell proliferation in a 7-day treatment course (FIGS. 2B and 11B).

### Example 3: Genetic Depletion of PDPK1 Sensitizes NRAS Mutant Melanoma Cells to MEKi

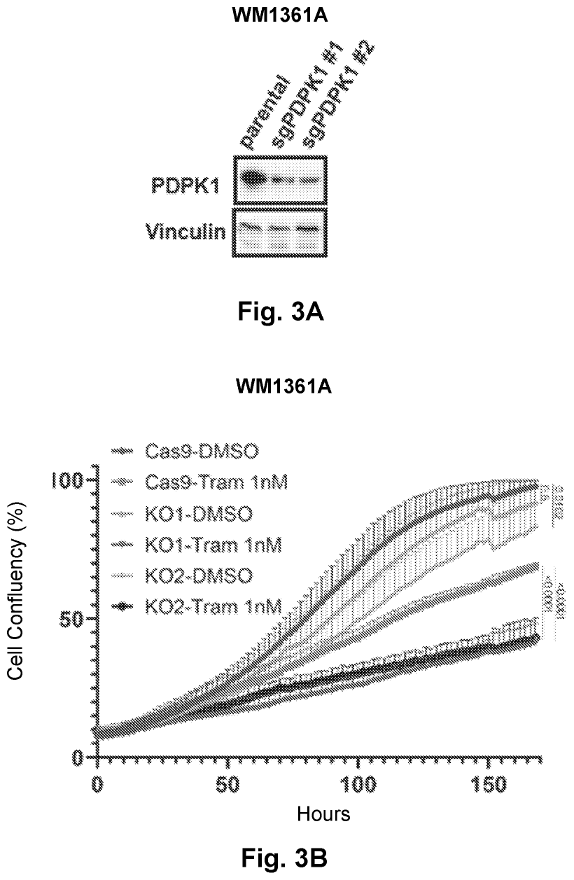

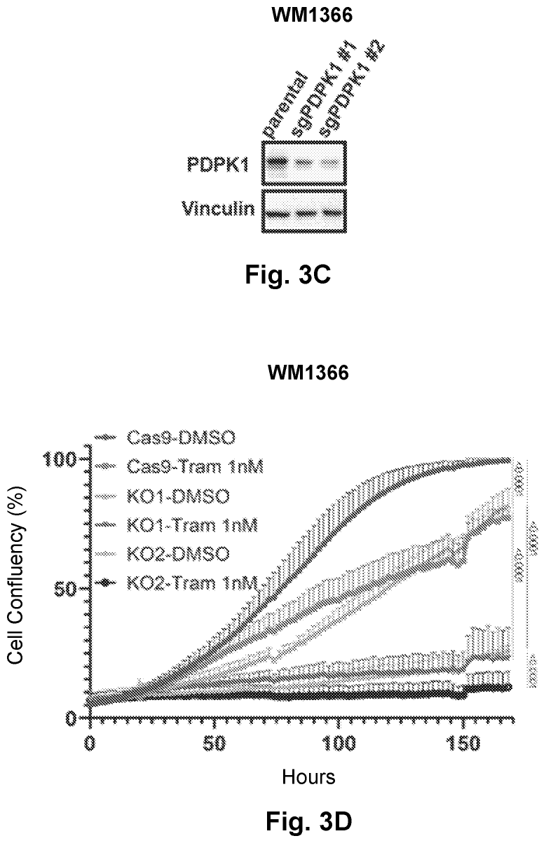

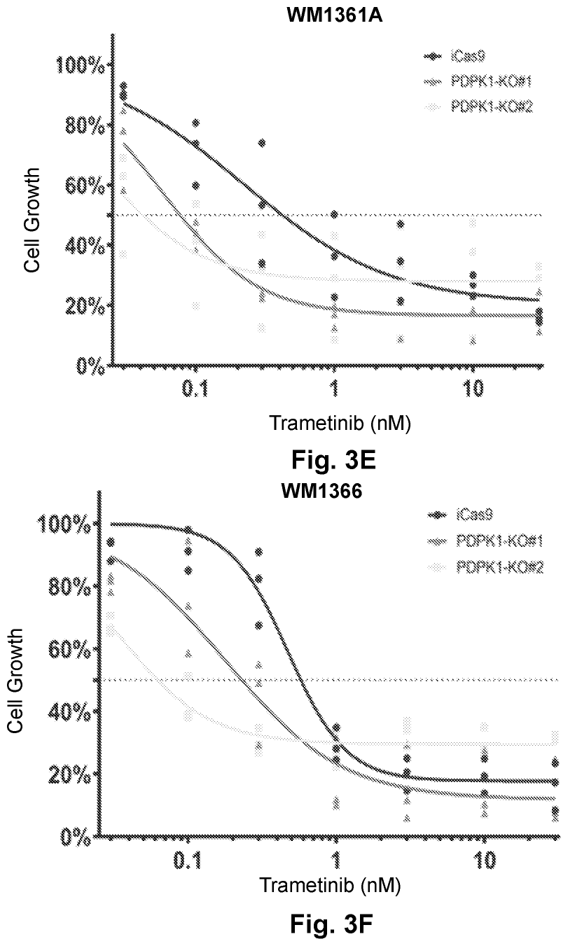

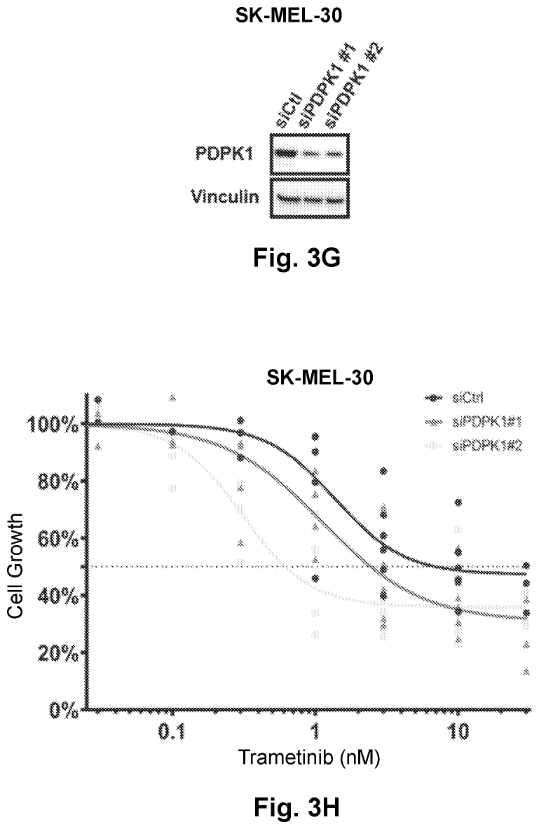

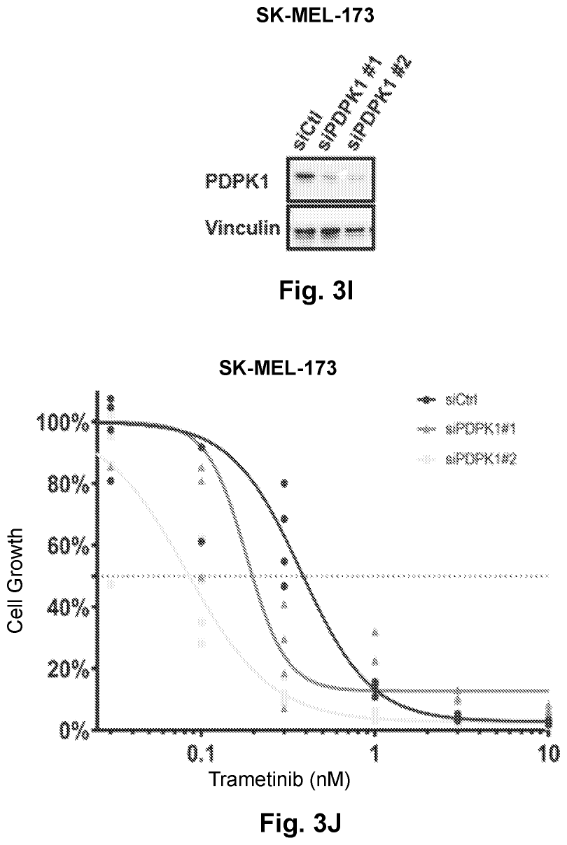

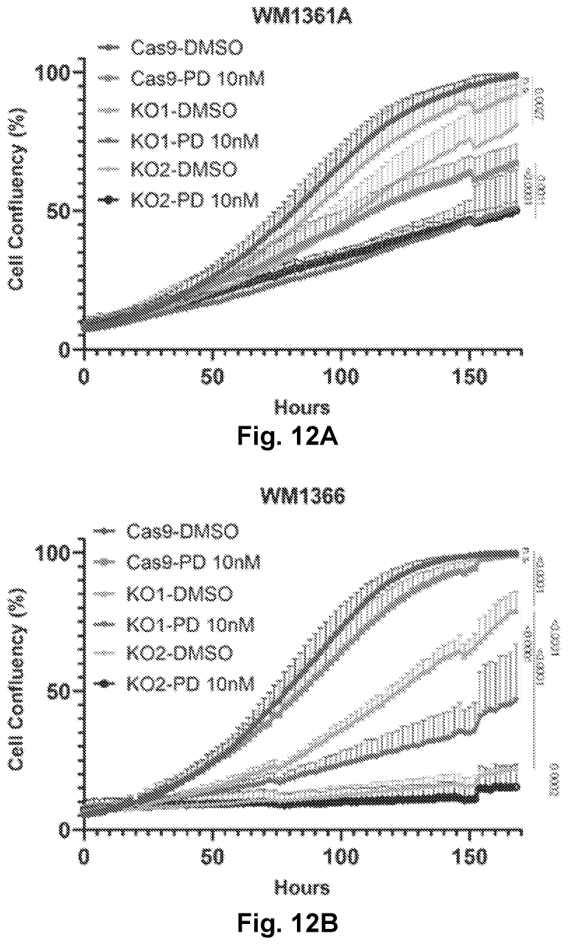

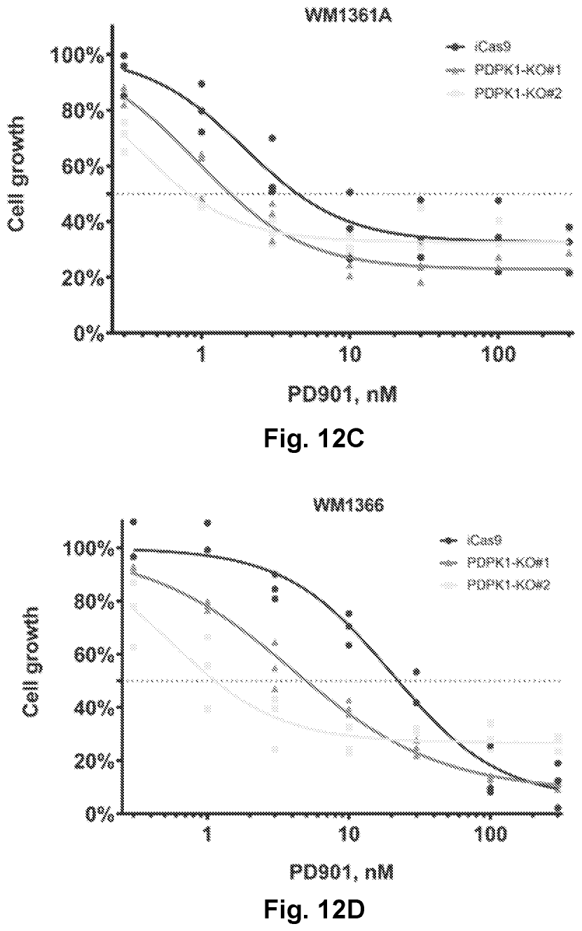

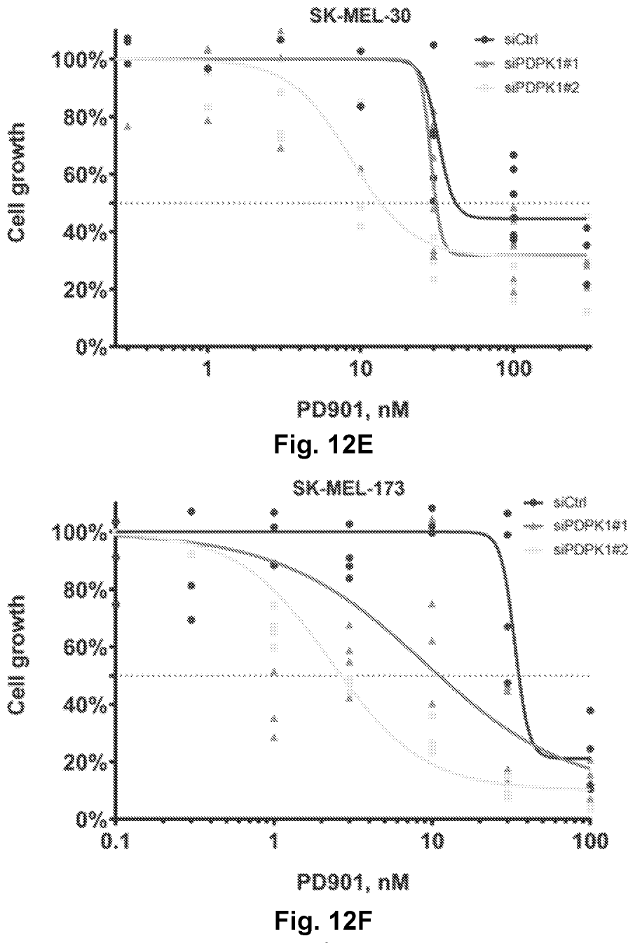

To test whether the synergistic effects of GSK2334470 was PDPK1-dependent, the present study generated PDPK1 knockout clones using high gene-editing efficiency clones of WM1361A and WM1366 cells. Depletion of PDPK1 by two different sgRNAs in both cell lines (FIGS. 3A-3B) attenuated cellular proliferation (FIGS. 3C-3D). Moreover, combining PDPK1 depletion with trametinib (FIGS. 3C-3D) or PD901 (FIGS. 12A-12B) effectively inhibited cell proliferation. PDPK1 depletion decreased the half-maximum inhibitory concentration of trametinib (FIGS. 3E-3F) and PD901 (FIGS. 12C-12D) in WM1361A and WM1366 by approximately 4-fold. Next, the present study sought to corroborate the results with siRNA-based PDPK1 knockdown (FIGS. 3G and 3I). Similar to findings with PDPK1 knockout, PDPK1 knockdown decreased the half-maximum inhibitory concentration of trametinib (FIGS. 3H and 3J) and PD901 (FIGS. 12E-12F).

### Example 4: Combined Inhibition of PDPK1 and MEKi Represses Xenograft Tumor Growth

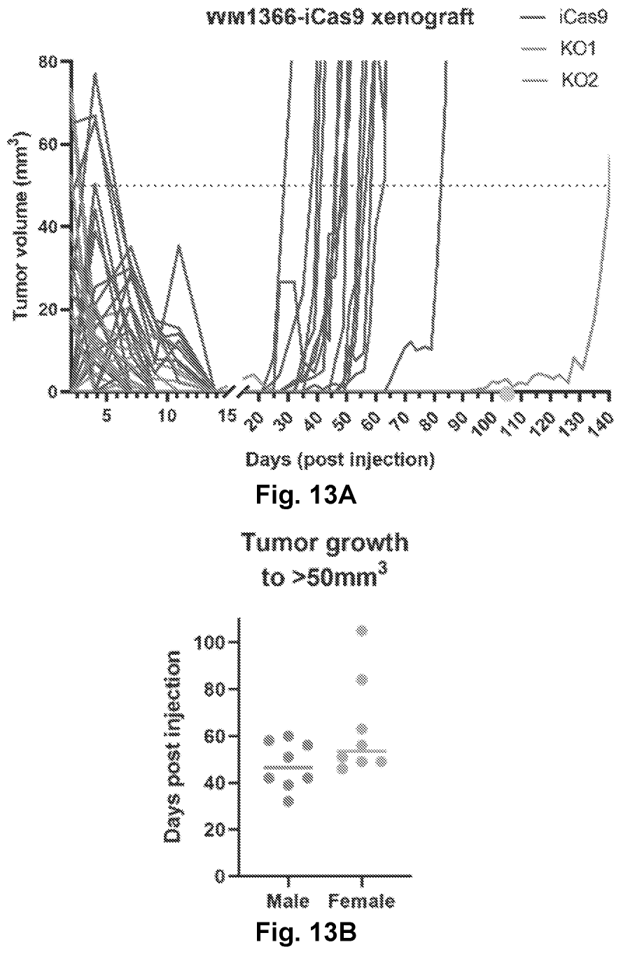

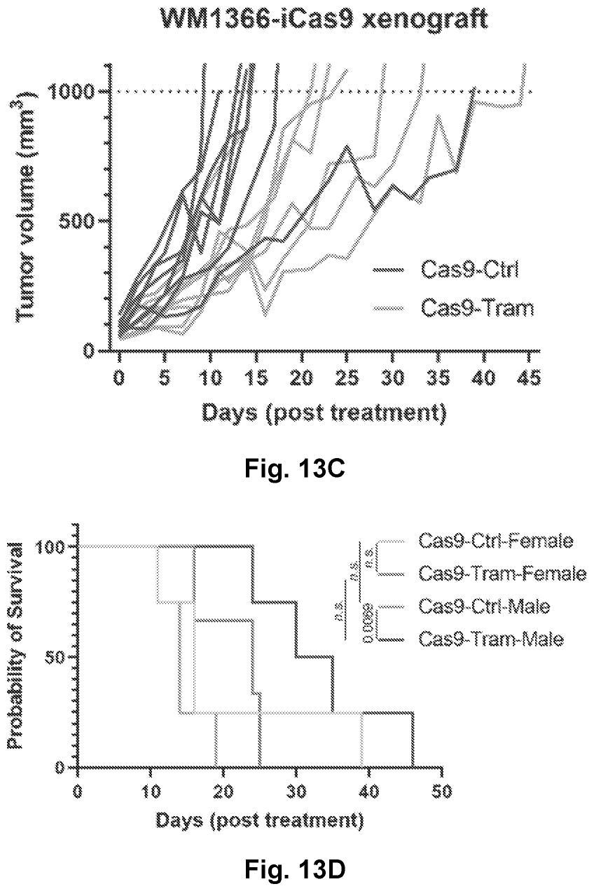

To investigate the role of PDPK1 in tumor growth in vivo, xenograft studies were performed with the parental Cas9-expressed clone and two WM1366 clones, in which PDPK1 was depleted by two individual sgRNAs (FIG. 3B). Mice were treated when tumor sizes reached 50 mm³ (FIG. 13A). Although basal tumor growth was slightly slower in female mice, the difference did not reach significance (p=0.0832) (FIG. 13B). Tumor growth was delayed in mice on a diet of trametinib-containing chow compared with mice on control chow (FIG. 13C). In mice injected with PDPK1 knockout cells, only one mouse (1/16) formed a visible tumor and no mice injected with K02 cells formed tumors after 3 months (FIG. 13A). No significant sex disparity was found during treatment (FIG. 13D). These data indicate that both PDPK1 depletion and MEK inhibition individually can reduce the growth of NRAS mutant melanoma xenografts.

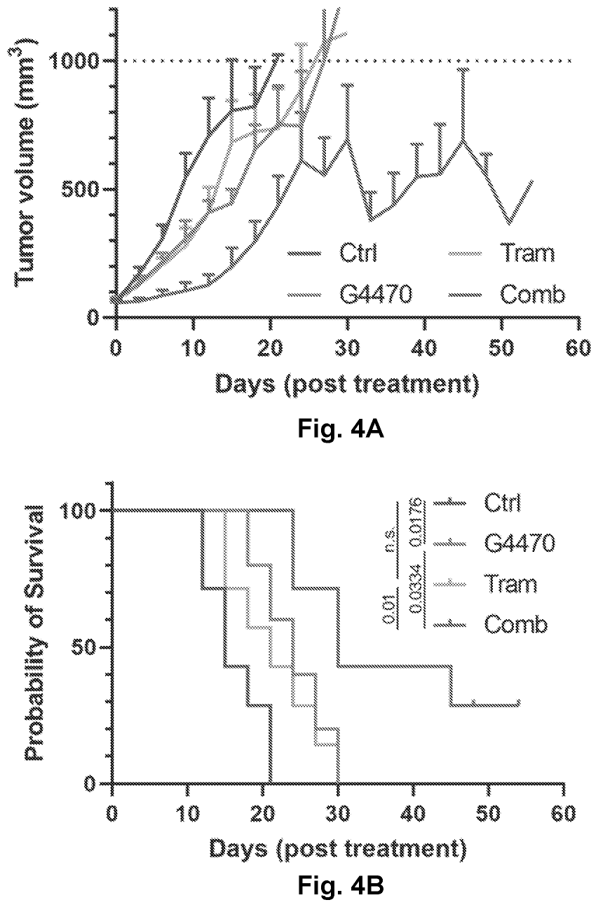

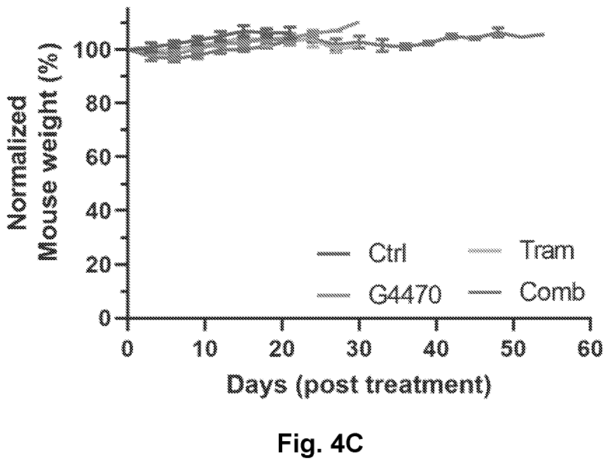

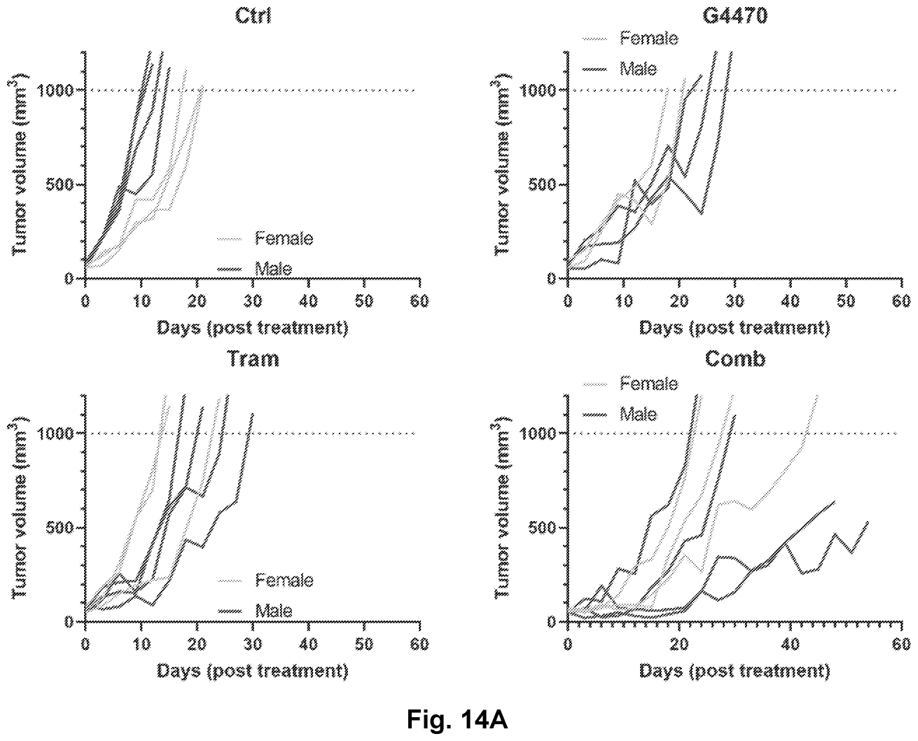

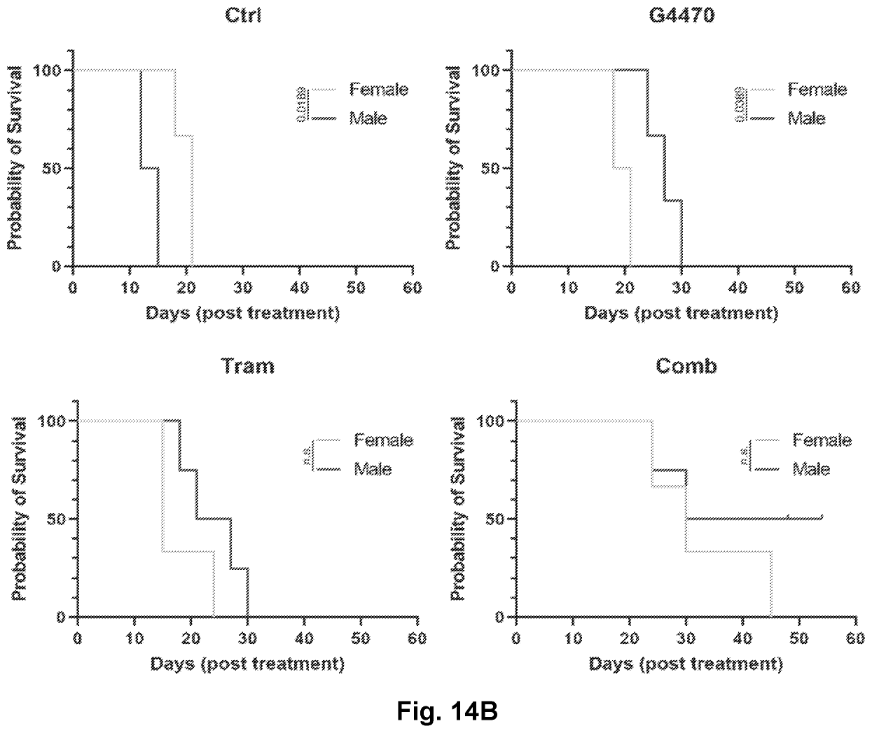

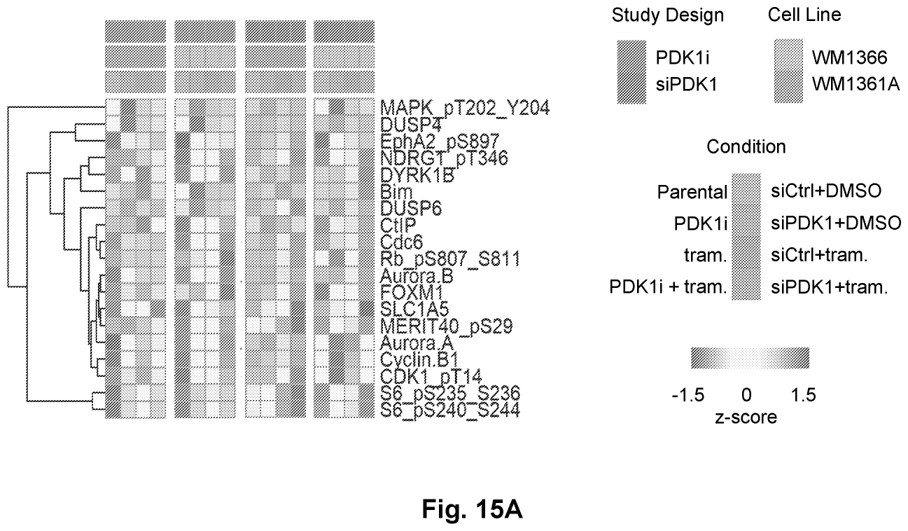

The present study further tested the therapeutic efficacy of combined MEKi and PDPK1i in vivo by utilizing xenograft models of SK-MEL-30 cells. Either PDPK1 or MEK inhibition suppressed tumor growth modestly, but the combinatorial treatment exerted a more profound suppression and a more prolonged survival than either treatment alone (FIGS. 4A and 4B). Moreover, the mice under treatments did not show significant weight loss in up to 54 days (FIG. 4C), suggesting tolerability. There was no significant difference between female mice and male mice in response to MEKi or PDPK1i+MEKi, whereas the female mice showed reduced tumor growth in control group (FIGS. 14A-14B), which may be due to SK-MEL-30 cells being derived from male patients and the induced immune responses against Y antigen. Nevertheless, these data suggest that PDPK1i+MEKi enables synergistic inhibition of the tumor growth of NRAS mutant melanoma.

### Example 5: Inhibition of PDPK1 Signaling and MEK Signaling Induces Pyroptosis

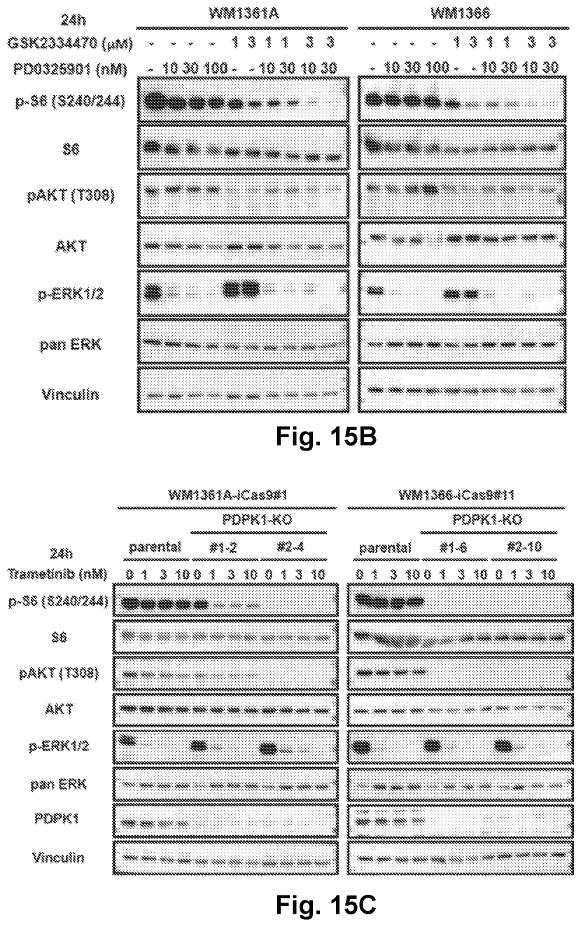

To gain greater insight into the effects of combined inhibition, reverse phase protein array (RPPA) of NRAS mutant melanoma cells treated with MEKi alone, PDPK1i alone, or the combination was performed. Samples from PDPK1 knockdowns treated +/− trametinib were also included. Since PDPK1 is a critical player in the PI3K-AKT pathway and the PI3K-AKT pathway and MAPK pathway merge to regulate the mTOR pathway, the present study analyzed the phosphorylation of S6. PDPK1i alone moderately decreased phosphorylation of S6 (p-S6) and the addition of trametinib led to further reductions p-S6 in W1366 and WM1361A cells (FIG. 15A). Using a second MEKi, PD0325901 and sgRNAs for PDPK1 depletion, the present study similarly found enhanced reduction of p-S6 (FIG. 15B-15C). Based on RPPA data, the pro-apoptotic regulator Bim was also affected by the combination. Proteins involved in cell cycle regulation were other targets of PDPK1i+MEKi with levels of p-RB (S807/S811), cyclin-B1 and Forkhead box protein M1 (FOXM1) being uniformly diminished upon both pharmacological and genetic inhibition of PDPK1 in conjunction with trametinib treatment (FIG. 15A).

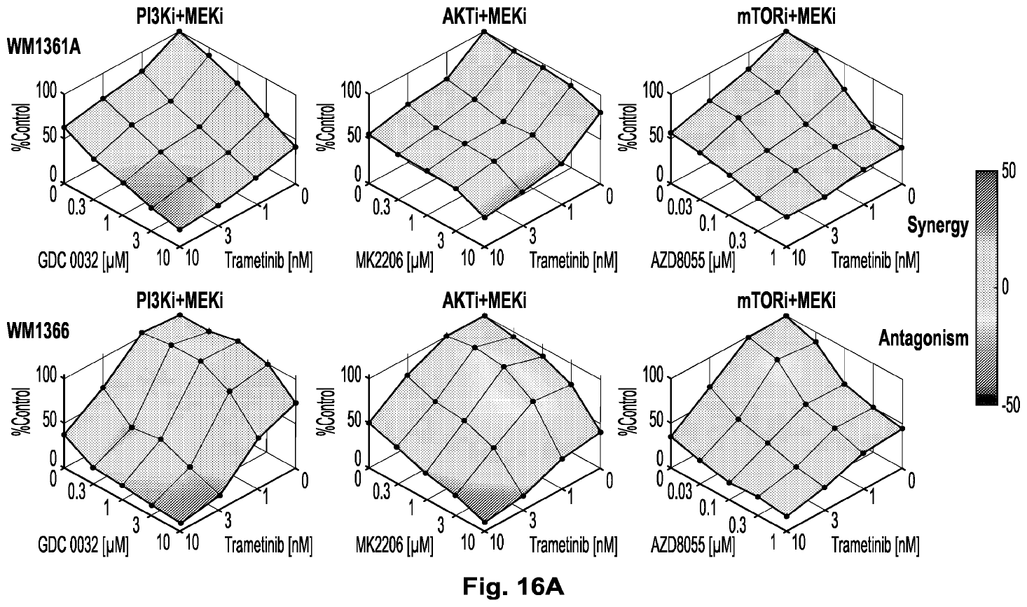

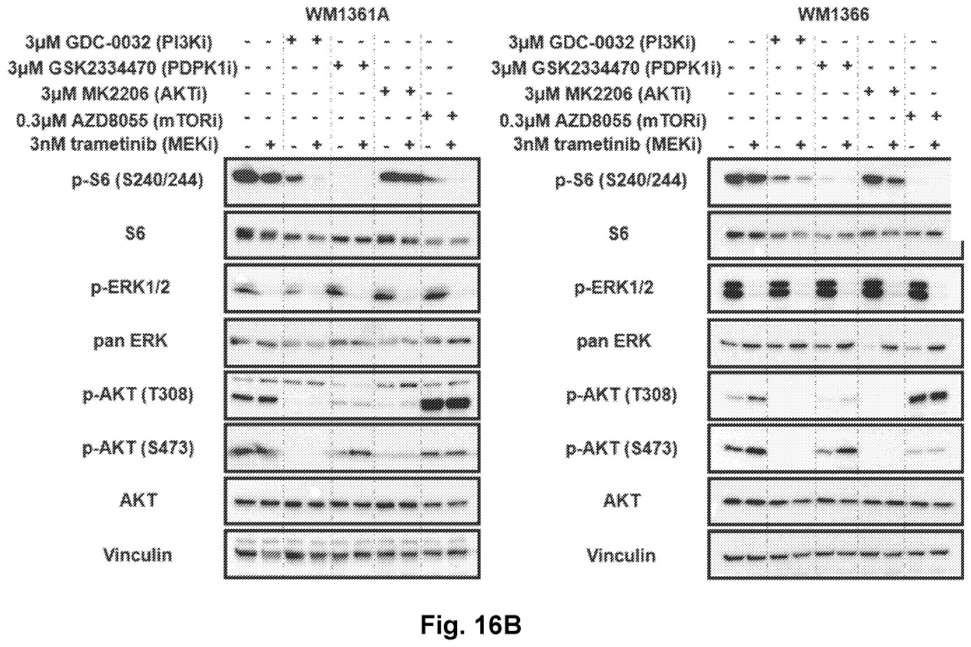

Next, the present study focused on targeting proteins within the PI3K-AKT-mTOR signaling axis. Whether PI3K, AKT, and/or mTOR inhibitors showed synergistic effects with MEKi was tested. PI3Kα/δ/γ inhibitor (PI3Ki) (GDC-0032) showed synergy with trametinib comparable to those with PDPK1i (FIG. 16A versus FIG. 2A). On the other hand, pan AKT inhibitor (AKTi) (MK-2206 2HCl) and mTORC1/2 inhibitor (mTORC1/2i) (AZD8055) showed reduced synergy and even antagonistic effects with MEKi (FIG. 16A). These data suggested that even though all these inhibitors target members of the PI3K-AKT-mTOR signaling axis (FIG. 16B), their combination with MEKi resulted in reduced synergy from the upstream to the downstream of the signaling axis as the order of PI3K-PDPK1-AKT-mTOR (FIG. 16A versus FIG. 2A).

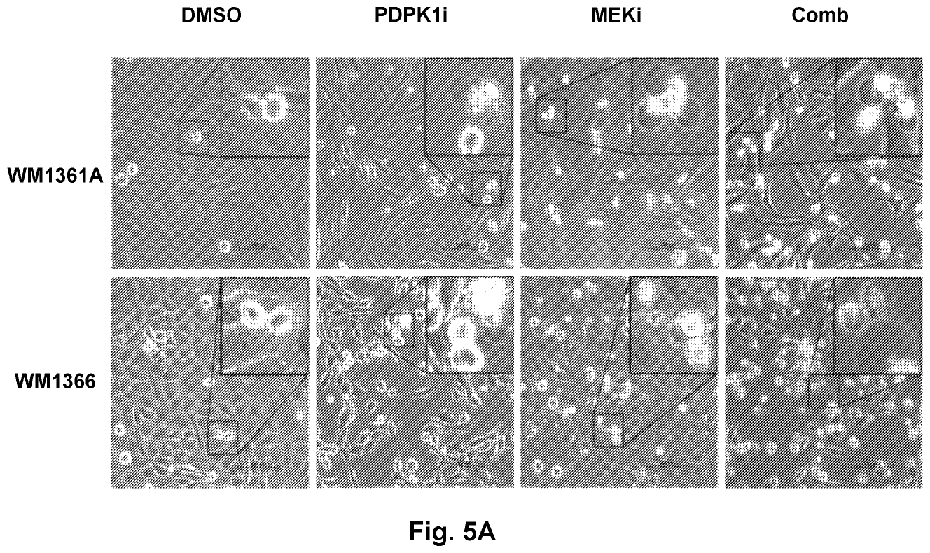

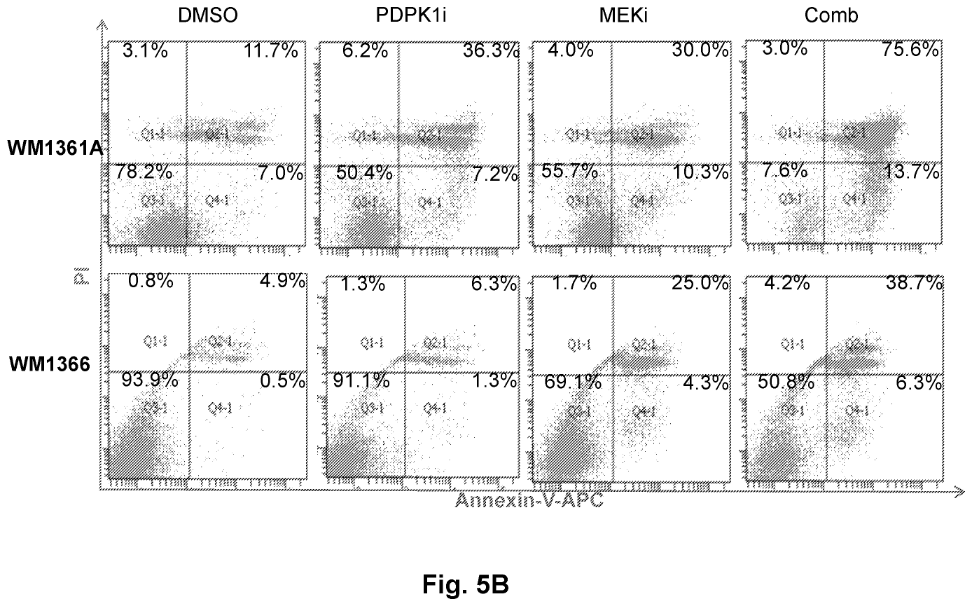

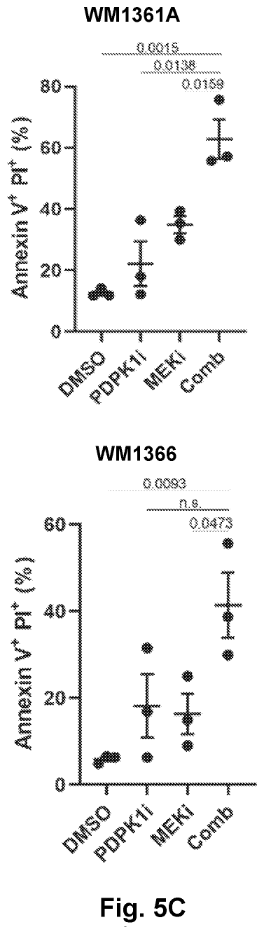

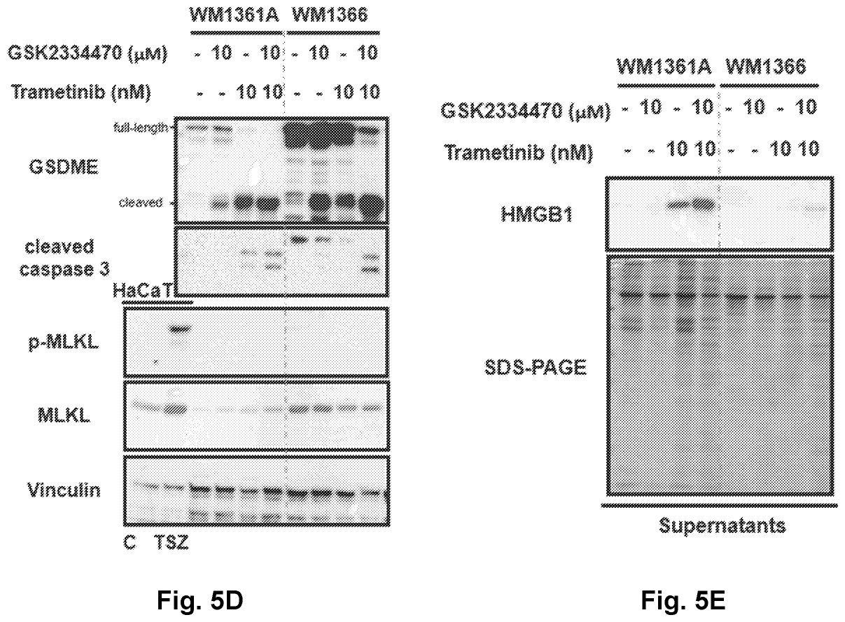

The present study further investigated the mechanism of cell death induced by combinatorial inhibition. Protrusions from the plasma membrane, a morphological feature of pyroptosis, were observed in cells treated with PDPK1i+MEKi (FIG. 5A). Moreover, a majority of dead cells after combined treatment exhibited double positivity for annexin-V and propidium iodide (PI), and only a relatively small portion of cells were apoptotic, as judged by a annexin-V-positive but PI-negative population (FIGS. 5B and 5C). PDPK1i+MEKi induced more cleaved caspase-3 associated with the cleavage of GSDME, an additional pyroptotic marker^{43,44} (FIG. 5D). Combined PDPK1i+MEKi treatment enhanced the release of HMGB1 to a greater extent than either PDPK1i or MEKi alone (FIG. 5E). The present study did not detect phosphorylated MLKL (p-MLKL), a marker of necroptosis (FIG. 5D). This finding is likely due to that melanoma cells seldom exhibit necroptosis due to the deficiency of RIPK3. Together, the results suggest concurrent targeting of PI3K-AKT and MEK-ERK1/2 has consequences not only for pathway inactivation but also for the induction of pyroptotic cell death.

### Example 6: Immune Responses Play a Critical Role in the Efficacy of Combined Inhibition

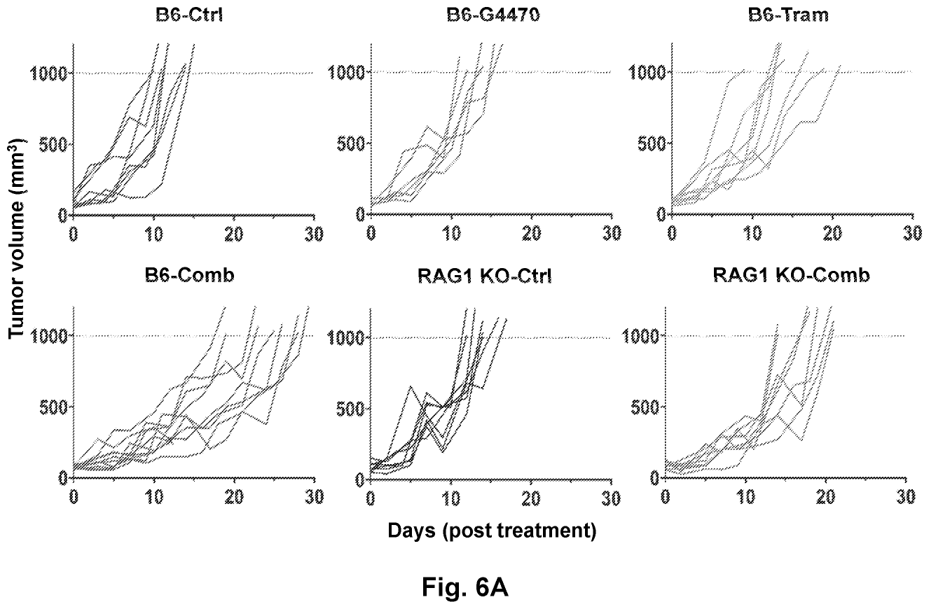

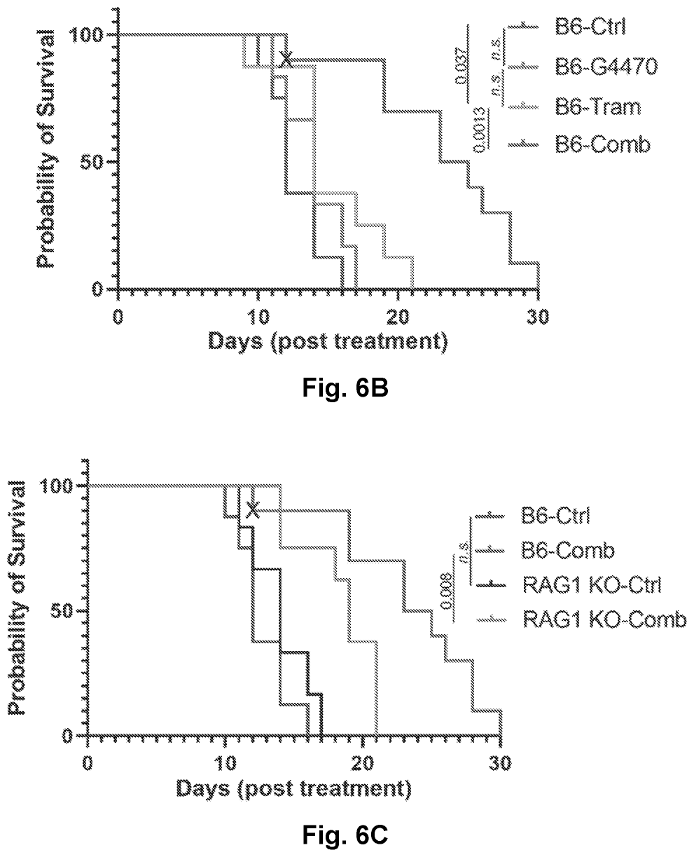

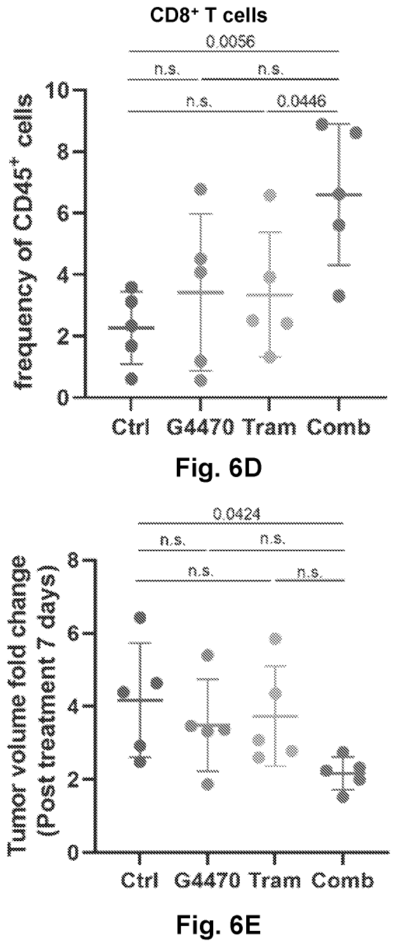

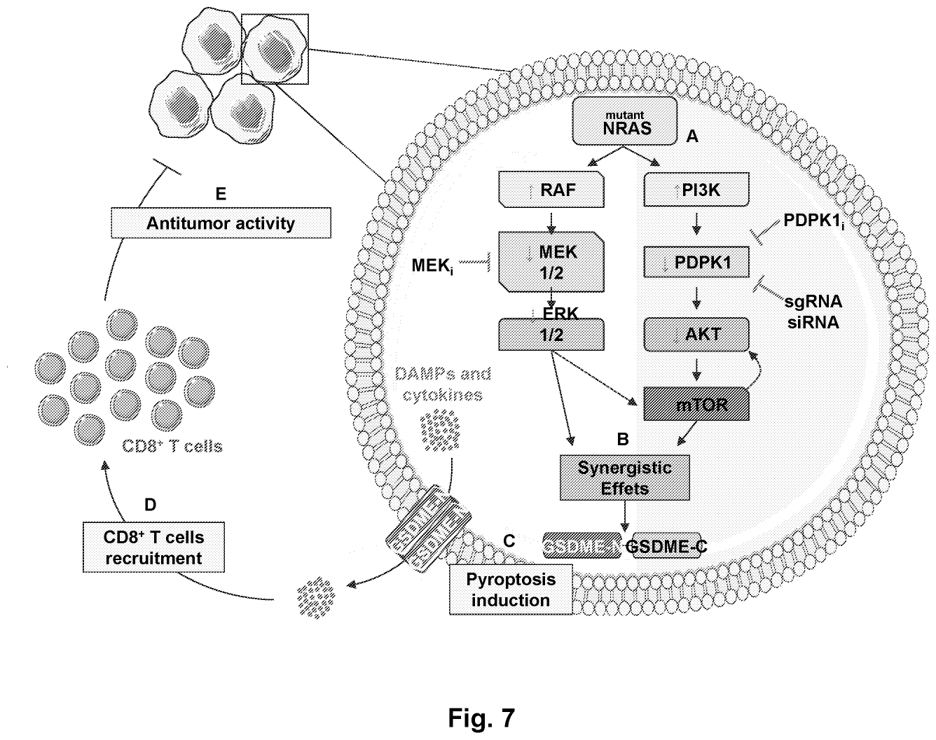

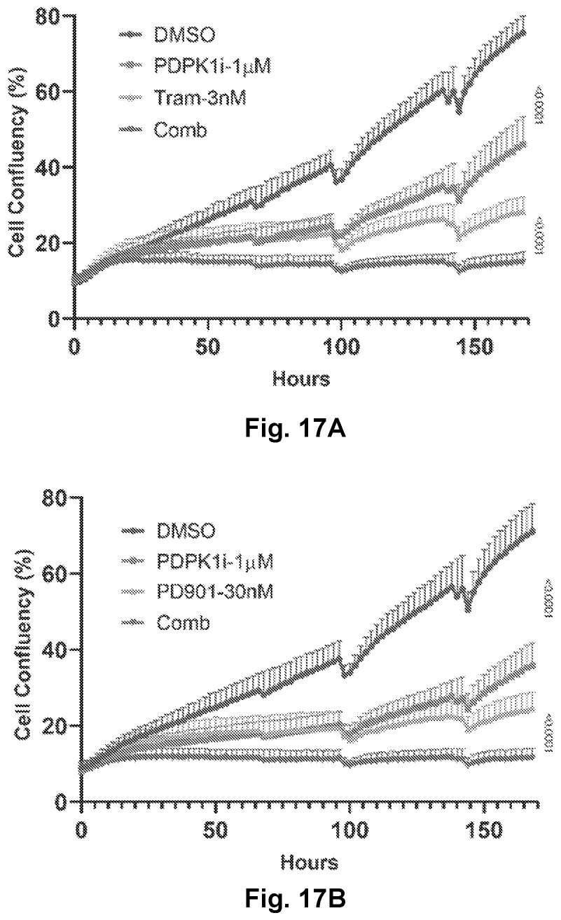

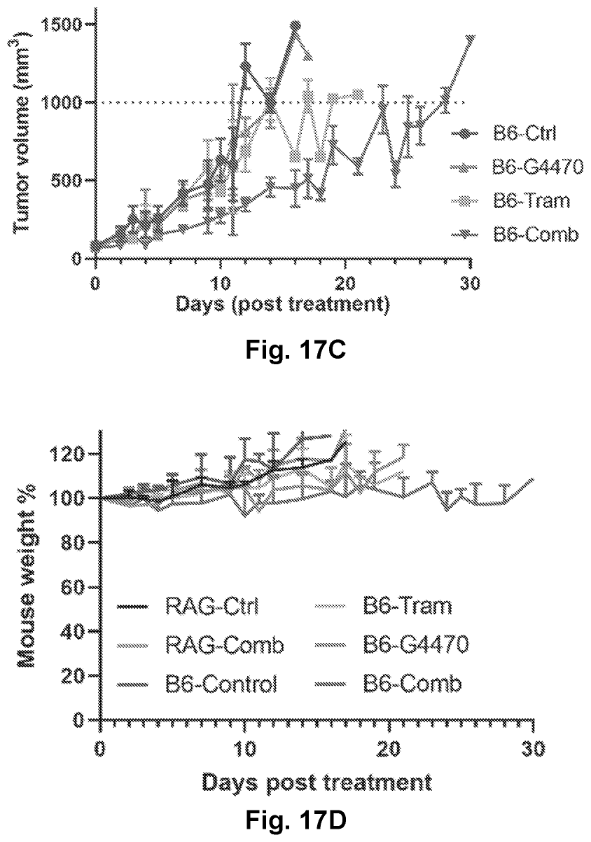

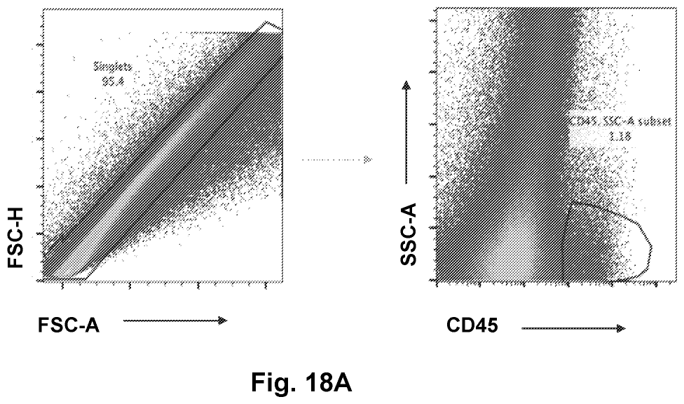

To investigate the functional contribution of the immune system to PDPK1i+MEKi therapeutic efficacy, the present study compared tumor responses to treatment in Nras^{Q61K} mouse melanoma isografts of 1014 cells in either immunocompetent (C57BL/6, B6) mice or syngeneic immunodeficient (B6.129S7-Rag1tm1Mom/J, RAG1 KO) mice. In vitro, PDPK1 and MEK inhibition individually suppressed 1014 cell proliferation and the combination led to a more profound suppression (FIGS. 17A-17B). In B6 mice in vivo, PDPK1 inhibition was not therapeutically effective whereas MEKi showed a modest delay of tumor growth (FIGS. 6A-6B and 17C). However, the combined inhibition significantly reduced tumor growth and prolonged mouse survival when compared with MEKi (FIGS. 6A-6B and 17C). In RAG1 KO mice, the combined inhibition also reduced tumor growth and prolonged mouse survival, however, the combination seemed to have higher efficacy in B6 mice (FIGS. 6A and 6C). Importantly, combined treatment did not result in significant weight loss in up to 30 days (FIG. 17D).

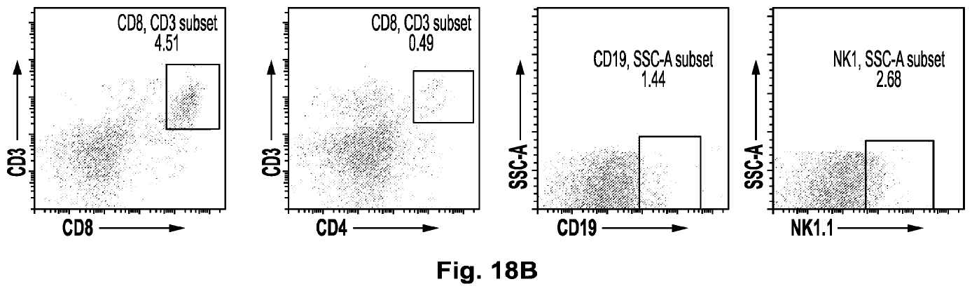

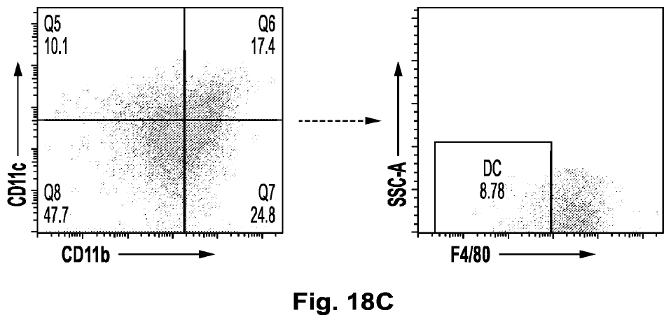

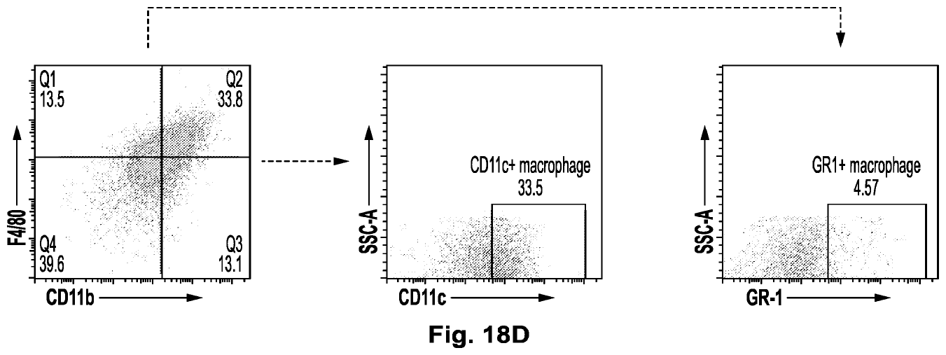

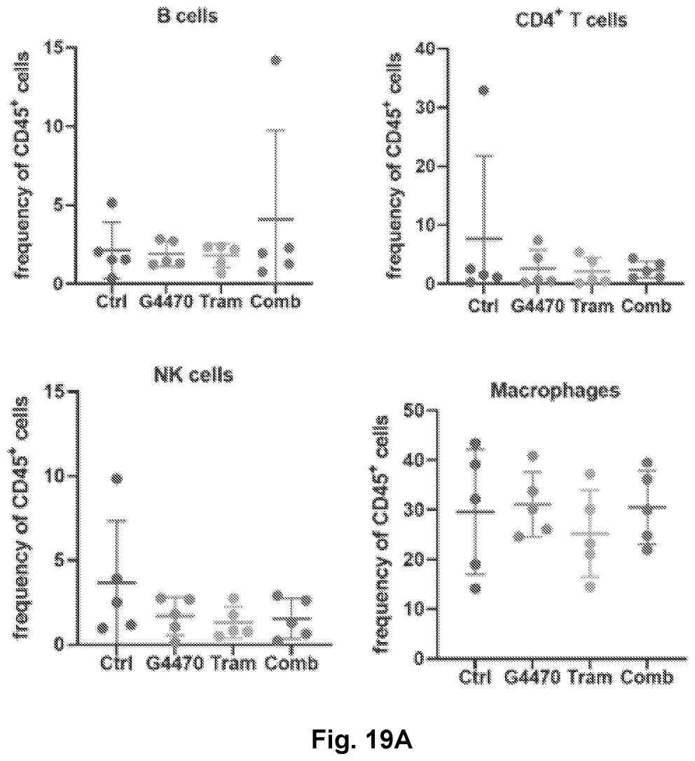

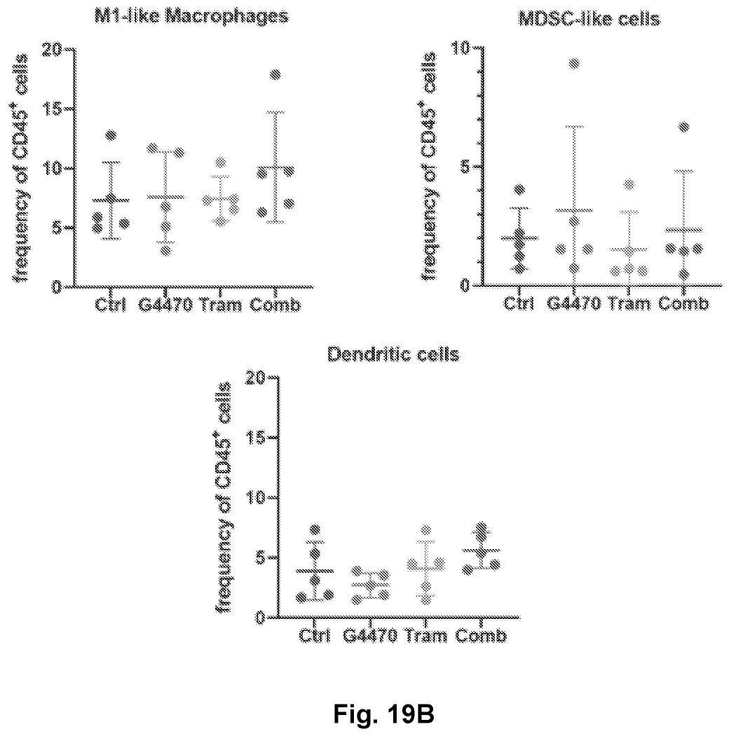

To examine the immune responses occurring in mice treated with PDPK1i, MEKi, and the combination of drugs, 1014 tumors were harvested from mice on day 7 post treatment and immune cells were enriched from tumor homogenates using density gradient media. The percent of T cells, B cells, NK cells, dendritic cells, and macrophages making up the immune compartment of the tumor were analyzed (FIGS. 18A-18D). The frequency of CD8⁺ T cells within the CD45⁺ cell compartment was significantly increased in the combination group over the control group, with the MEKi and PDPK1i groups exhibiting an intermediate effect (FIG. 6D). Dendritic cells and M1-like macrophages showed slight increase in the combination group but the difference did not reach significance (FIGS. 19A-19B). These data suggest that PDPK1i+MEKi increased the percentage of CD8⁺ T cells infiltration and that the adaptive immune response significantly contributed to therapeutic efficacy.

### Example 7

The present study identified novel therapeutic targets that will potentially enhance MEK inhibitor efficacy in NRAS mutant melanoma, and validated the anti-tumor efficacy of combined inhibition in vivo, which signal promising translational application.

One target identified found in the screen is PDPK1. PDPK1 is the master kinase downstream of PI3K for the activation of key AGC kinases and oncogenic signaling pathways such as the AKT, PKC, p70S6K, SGK, PLCγ1, and Plk/cMyc pathways. However, there is currently no ongoing clinical trial utilizing PDPK1i.

NRAS mutations activate both MAPK pathway and PI3K-AKT-mTOR pathway. However, dual MEK/AKT inhibition did not yield clinical benefit in metastatic NRAS mutant melanoma and the data has also shown that dual AKT/MEK inhibition showed a more modest synergy comparing with dual PI3K/MEK inhibition (FIG. 13A) or dual PDPK1/MEK inhibition (FIG. 2A) in vitro.

In NRAS mutant melanoma cells, PDPK1i+MEKi treatment was able to induce hallmark features of pyroptosis such as cleavage of GSDME and release of HMGB1 (FIG. 5), which is critical in inducing anti-tumor immune responses in BRAF mutant melanomas treated with BRAF and MEK inhibitors. Notably, a small fraction of tumor cells undergoing pyroptosis are sufficient to trigger durable immune response against the tumor.

The data also indicate that the adaptive immune response constitutes a critical portion of the full efficacy of PDPK1i+MEKi (FIGS. 6C and 16D) and that PDPK1i+MEKi treatment significantly increased the percent of intratumoral CD8+ T cells (FIG. 6E). It's possible that pyroptosis is involved in the changes in the CD8+ T cell responses seen in the PDPK1i+MEKi treated tumors as well.

### Example 8: Material and Methods

### Cell Culture

Human WM1361A and WM1366 melanoma cell lines were cultured in MCDB153 (Sigma) with 2% FBS, 20% Leibowitz L-15 medium, and 5 μg/mL insulin (WM medium). Human SK-MEL-30 and SK-MEL-173 melanoma cell lines were cultured in RPMI-1640 with 10% FBS. Mouse 1007 and 1014 melanoma cell lines were cultured in Ham's F12 medium supplemented with 10% fetal bovine serum, 5 mM L-glutamine. The human immortalized keratinocytes HaCaT cells were cultured in Dulbecco's modified Eagle's medium supplemented with 10% fetal bovine serum and 1% penicillin/streptomycin. All cells were cultured at 37° C. in a humidified atmosphere containing 5% CO2. WM1361A was kindly donated by Dr. Meenhard Herlyn (Wistar Institute, Philadelphia, PA) in 2005. WM1366 was purchased from Rockland Immunochemicals, Inc. in 2019. SK-MEL-30 and SK-MEL-173 were kindly donated by Dr. David Solit (Memorial Sloan-Kettering Cancer Center, New York, NY) in 2015. 1007 and 1014 were kindly donated by Dr. Lionel Larue (Equipe Labellisée Ligue Contre le Cancer, Orsay, France) in 2018. Cell lines were STR analyzed, confirmed for NRAS/Nras mutation, and tested to be mycoplasma-free during experiments. For cell proliferation and drug response, cells were monitored by IncuCyte® (Essen Bioscience, Ann Arbor, MI) every 2 hours.

Generation of WM1361A-iCas9 sgRNA Library

Lenti-iCas9-neo was a gift from Dr. Qin Yan (Addgene plasmid #85400) (Cao et al. Nucleic Acids Res 44, 2016). Lentivirus based on Lenti-iCas9-neo were produced in Lenti-X 293T cells (Takara Bio Cat No. 632180) and infected WM1361A cells with polybrene (4 μg/ml, Santa Cruz, sc-134220). After overnight culture, refresh medium with doxycycline (1 μg/ml) was replaced. GFP-positive cells were sorted as individual clones on 96-well plates.

Gene-editing efficiency of each clone is tested by abolishing GFP or BFP signaling with corresponding sgRNAs. In brief, pU6-sgRNA EF1Alpha-puro-T2A-BFP was a gift from Jonathan Weissman (Addgene plasmid #60955) (Gilbert et al. Cell 159, 647-661, 2014) and encodes sgRNA against GFP (renamed as pU6-sgGFP-BFP). The plasmid was digested by BstXI & BlpI and cloned with inserted sequence which encodes sgRNA against BFP. The inserted sequence was annealed from sgBFP-F 5′ ttgGTCACCACATACGAAGACGGgtttaagagc 3′ (SEQ ID NO:1) and sgBFP-R 5′ ttagctcttaaacCCGTCTTCGTATGTGGTGACcaacaag 3′ (SEQ ID NO:2). The resulted plasmid was named as pU6-sgBFP-BFP. WM1361A-iCas9 clones were infected by lentivirus based on pU6-sgGFP-BFP or pU6-sgBFP-BFP and selected by puromycin (Gibco A11138-03) for 7 days. After treatment with or without doxycycline for 3 days, parental cells and infected cells were analyzed by flow cytometry. Clones with gene-editing efficiency above 90% in both sgRNAs were considered as candidates for the pooled screen.

The Guide-it CRISPR Genome-Wide sgRNA Library System (Cat. No. 632646) was purchased from Takara Bio. The production of lentivirus containing sgRNA library followed the manufacturer's protocol. WM1361A-iCas9 #1 cells were validated by STR analysis and were selected for the screen as the clone with the highest mCherry-positivity infected with same amount of virus containing sgRNA library among several clones. Around 1.8×108 cells were seeded into 6 well plates one day prior to infection. Then, 2.34×108 cells were infected with the virus with 4 μg/ml of polybrene, spun at 1,200×g for 90 min at 4° C., and replaced with fresh medium overnight. After 3 days, half of the cells were harvested and analyzed by flow cytometry. The remaining cells were treated with hygromycin (50 μg/ml, InvivoGen, BGG-41-02) for 10 days. Three samples were harvested at 10 days, 17 days and 24 days post infection.

Negative and Positive Screen of WM1361A-iCas9 sgRNA Library with Trametinib

On 24 days post infection, 3×108 cells with sgRNA library were divided into 3 arms: DMSO as vehicle control, 0.01 nM trametinib as a negative screen and 20 nM trametinib as a positive screen. The cells were passaged twice per week and maintained at 1×108 cells for each group during passage. 20 nM trametinib were treated for 2 weeks whereas DMSO and lower concentration of trametinib were treated for 4 weeks (0.01 nM trametinib for 3 weeks and 0.02 nM trametinib for further 1 week). 1×10⁸ cells were harvested and stored at −80° C. for each arm every 7 days.

Genomic DNA was extracted with Wizard® Genomic DNA Purification Kit (Promega, A1120). DNA fragments containing coding sequences of sgRNAs were amplified with Guide-It™ CRISPR Genome-Wide Library PCR Kit (Takara Bio, Cat. No. 632646) according to manufacturer's manual and purified in agarose gel. The sequencing libraries were prepared using the Guide-it CRISPR Genome-Wide sgRNA library NGS Analysis Kit (Takara Bio, Cat. No. 632647) following manufacturer's protocol. The normalized final libraries were sequenced on Illumina NextSeq 500 platform using 75 bp v2.5-chemistry at 1.8 pM final concentration with 20% PhiX (Illumina).

Bcl2fastq was used to generate raw fastq files. PoolQ (v3.0.5) was performed to count the number of 20-base sgRNA library barcodes for each sample barcode, while allowing for a single base mismatch [https://portals.broadinstitute.org/gpp/public/software/poolq]. A custom script was used to filter control guides and normalize data to counts per million. STARS (v1.3) (Doench et al. Nat Biotechnol 34, 184-191, 2016) was used with a 10% threshold and exclusion of the first guide to score genes, and 1,000 iterations to develop a null distribution. The Therapeutic Targets Database (TTD) v7.1.01 (Wang et al. Nucleic Acids Res 48, D1031-D1041, 2020) was used to identify the names and highest clinical status of drugs for targeting enriched genes.

### Transient and Stable Expression

For siRNAs, cells were transfected with ON-target control (5′ UGGUUUACAUGUCGACUAAUU 3′, Dharmacon D-001810-01, SEQ ID NO:3), PDPK1 #1 (5′ CAAGAGACCUCGUGGAGAAUU 3′, Dharmacon D-003017-05, SEQ ID NO:4) or PDPK1 #2 (5′ GACCAGAGGCCAAGAAUUU 3′, Dharmacon D-003017-06, SEQ ID NO:5) siRNAs at a final concentration of 25 nM using Lipofectamine RNAiMAX (Invitrogen). For drug treatment, cells were treated with drugs in fresh media post-transfection 24 hours.

For PDPK1 sgRNAs, the inserted sequences were annealed from sgPDPK1-IF 5′ CACCGCAAGTTTGGGAAAATCCTTG 3′ (SEQ ID NO:6) plus sgPDPK1-1R 5′ AAACCAAGGATTTTCCCAAACTTGC 3′ (SEQ ID NO:7), or sgPDPK1-2F 5′ CACCGCCCGCTCTCTGGTTACATAG 3′ (SEQ ID NO:8) plus sgPDPK1-2R 5′ AAACCTATGTAACCAGAGAGCGGGC 3′ (SEQ ID NO:9) and were inserted into pLVXS-sgRNA-mCherry-hyg vector according to the manufacturer's protocol (Takara Bio). Then WM1361A-iCas9 #1 or WM1366-iCas9 #11 cells were infected with lentivirus expressing sgRNAs and selected by hygromycin (InvivoGen). Individual clones were further validated by immunoblot.

### Reverse-Phase Protein Array (RPPA) Analysis

WM1361A and WM1366 cells were treated with vehicle control, PDPK1i (GSK2334470), MEKi (trametinib) or combined PDPK1i and MEKi for 48 hours. WM1361A and WM1366 cells were also transfected with ON-target control siRNAs or PDPK1 siRNAs, and then treated with or without trametinib for further 48 hours. Cells were harvested, lysed and analyzed as described in Tibes et al. (Mol Cancer Ther 5, 2512-2521, 2006). Samples were normalized as described at https://www.mdanderson.org/research/research-resources/core-facilities/functional-proteomics-rppa-core/faq.html. Differential expression analysis was performed between each combination treatment group against the relevant control for each cell line using the limma package (v3.44.3) (Shi et al. Nucleic Acids Res 38, e204, 2010). A Benjamini-Hochberg False Discovery Rate (BHFDR) value <0.05 was used to identify significant antibodies. Antibodies that were significant in at least three comparisons were included.

### Immunoblot Analysis and Cell Supernatant Collection

After washing with ice-cold PBS, cultured cells were scraped and lysed in Laemmli sample buffer or RPPA lysis buffer. Samples were boiled for 5 min and used in SDS-PAGE. After standard western blot procedures, the blots were developed using the ChemiDoc MP Imaging System (Bio-Rad) with HRP-conjugated secondary antibodies (CalBioTech) and chemiluminescence substrate (Thermo Scientific). For tumor samples, tumors were excised and lysed in 1% SDS containing a protease inhibitor cocktail (Roche) and a phosphotase inhibitor cocktail (Roche) followed by sonication. After centrifuging for 10 min at 20,000×g, the supernatants were combined with 4x Laemmli sample buffer, boiled and run via Western blotting as described above. The antibody for vinculin (sc-73614) was purchased from Santa Cruz Biotechnology. Antibodies for PDPK1 (#3062), p-AKT (S473) (#4060), p-AKT (T308) (#2965), AKT (#9272), p-ERK1/2 (T202/Y204) (#9101), p-S6 (S240/244) (#2215), S6 (#2217), caspase 3 (#9662), HMGB1(#6893), MLKL(#14993) and p-MLKL (S358) (#91689) were purchased from Cell Signaling Technology. The antibody for pan ERK (M12320) was purchased from Transduction. The antibody for GSDME (ab215191) was purchased from Abcam. Cell supernatants were harvested in the absence of FBS in culture medium to avoid distortion of SDS-PAGE. Cell debris was removed by a brief spin. Then cell supernatants were concentrated 1Ox with Amicon Ultra 10K (Sigma-Aldrich), and mixed with Laemmli sample buffer (Bio-Rad) for immunoblot. Protein gels were stained with Coomassie Brilliant Blue R-250.

### In Vivo Tumor Growth Studies:

Animal experiments were performed at Thomas Jefferson University in a facility accredited by the Association for the Assessment and Accreditation of Laboratory Animal Care and were approved by the Institutional Animal Care and Use Committee (protocol #1052). All mice were purchased from Jackson Lab at age of 4-5 weeks, housed for one week, and then numbered for further experiments. For tumor formation, various melanoma cells were trypsinized, washed twice with HBSS and injected into left flanks of mice subcutaneously in 100 μL HBSS. When tumors became palpable, the tumor volumes were tracked three times per week with a digital caliper using the following formula: volume=(length×width2)×0.52. Mouse weights were monitored once a week or three times per week during drug treatment. Mice were sacrificed when tumor volume reached 1,000 mm³.

For the WM1366-iCas9 xenograft model, eight male and eight female Nu/J mice (Homozygous, stock #002019) were injected with 5 million WM1366-iCas9 #11, WM1366-iCas9-sgPDPK1 #1 or WM1366-iCas9-sgPDPK1 #2 cells per mouse. When the tumor volume reached 50 mm³, sex-balanced animals were randomly assigned to two cohorts, fed with either vehicle control chow (AlN-76A diet) or MEKi chow (0.3 ppm trametinib) (Brighto, et al. Cancer Res 78, 542-557, 2018) which were made by Research Diets Inc.

For the SK-MEL-30 xenograft model, 24 male and 24 female Nu/J mice were injected with 10 million SK-MEL-30 cells per mouse. Two female mice died for a non-experiment-related reason before injection. When the tumor volume reached 50 mm³, sex-balanced animals were randomly assigned to four different cohorts, fed with either vehicle control chow or MEKi chow, and combined with mock (5% DMSO+40% PEG300+10% Tween-80+45% water as vehicle, 10 μl per gram mouse weight) injection or PDPK1i (GSK2334470 dissolved in DMSO as 200 mg/ml stock, formulated at 10 mg/ml in vehicle, 100 mg/kg in mouse) injection intraperitoneally every 3 days.

For the 1014 allograft model, female C57BL/6 (B6) mice (stock #000664) and B6.129S7-Rag1tm1Mom/J (Rag1 KO) mice (stock #002216) were injected with 1 million 1014 cells. For survival analysis, 32 B6 mice were randomly assigned to four different cohorts: control chow+mock injection, MEKi chow+mock injection, control chow+PDPK1i injection and MEKi chow+PDPK1i injection after the tumor volume reached 50 mm³. The intraperitoneal injections were performed three times per week. In addition, 16 Rag1 KO mice were randomly assigned to two different cohorts: control chow+mock injection and MEKi chow+PDPK1i injection after the tumor volume reached 50 mm³. For immunology analysis, 24 mice were randomly assigned to four different cohorts: control chow+mock injection, MEKi chow+mock injection, control chow+PDPK1i injection and MEKi chow+PDPK1i injection after the tumor volume reached 100 mm³, treated for 7 days and then sacrificed for further analysis.

### Flow Cytometry of Tumor-Infiltrating Lymphocytes (TIL)

For immunogenicity and TIL studies, tumors were removed from sacrificed mice and dissociated into single cell suspensions using the Mouse Tumor Dissociation Kit (Miltenyi Biotec, 130-096-730) on gentleMACS Octo Dissociator using C Tubes (Miltenyi Biotec) as manufacturer's manual. Leukocytes in tumor samples were further enriched with Lymphoprep (STEMCELL Technologies). Leukocytes obtained from homogenizing one spleen using a 70 μM nylon filter and a syringe plunger were used as an unstained control and single color controls. Cells were surface stained with the following conjugated antibodies from BioLegend: CD45.2 (clone 104) in BV421, CD3 (clone 17A2) in PE, CD8a (clone 53-6.7) in PE/Dazzle, CD4 (clone GK1.5) in BV605, NK1.1 (clone PK136) in PerCP-Cy5.5, CD11c (clone N418) in APC-Cy7, CD11b (clone M1/70) in BV650, CD19 (clone 2E7) in Alexa Fluor 700, F4/80 (clone BM8) in APC, and GR1 (clone RB6-8C5) in FITC. All samples were analyzed on the BD Celesta and data was analyzed using FlowJo.

### Annexin V/Propidium Iodide (PI) Staining

Cells were treated as indicated in each experiment. At the end of the treatment period, medium was collected to harvest floating cells, cells were washed with PBS, and PBS was collected to harvest the remaining floating cells. Attached cells were trypsinized, combined with floating cells, and spun down. Cell pellets were washed twice with PBS and then re-suspended in binding buffer containing 1:20 annexin V-APC (BD Pharmingen) and 0.2 mg/ml PI (Life Technologies). Samples were incubated at room temperature for 15 min protected from light, and annexin V-APC and PI fluorescence was analyzed on the BD Celesta.

### Statistical Analysis and Reproducibility

Individual in vitro experiments were performed three times unless otherwise indicated. p value is calculated by GraphPad Prism 9.0 with various tests indicated in Figure Legends. p value <0.05 was considered significant and the numbers are indicated in the figures. Results are expressed as mean or mean±SEM unless otherwise indicated. No animals were excluded from experiments. No statistical method was used to predetermine sample size. Sample size was chosen on the basis of literature in the field.

### Enumerated Embodiments

In some aspects, the present invention is directed to the following non-limiting embodiments:

Embodiment 1: A composition for treating, preventing, and/or ameliorating melanoma in a subject in need thereof, comprising: a MEK inhibitor, or a salt or solvate thereof, and at least one of a PDPK1 inhibitor, or a salt or solvate thereof, and a PI3K inhibitor, or a salt or solvate thereof.

Embodiment 2: The composition of Embodiment 1, wherein the melanoma is an NRAS mutant melanoma.

Embodiment 3: The composition of any of Embodiments 1-2, wherein the subject is a mammal.

Embodiment 4: The composition of any of Embodiments 1-3, wherein the subject is a human.

Embodiment 5: The composition of any of Embodiments 1-4, further comprises a pharmaceutically acceptable excipient or carrier.

Embodiment 6: The composition of any of Embodiments 1-5, wherein the MEK inhibitor comprises N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib) or N-[(2R)-2,3-dihydroxypropoxy]-3,4-difluoro-2-(2-fluoro-4-iodoanilino)benzamide (PD0325901), or a salt or solvate thereof.

Embodiment 7: The composition of any of Embodiments 1-6, wherein the composition comprises the PDPK1 inhibitor, or a salt or solvate thereof, and wherein the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or a salt or solvate thereof.

Embodiment 8: The composition of any of Embodiments 1-7, wherein the composition comprises the PI3K inhibitor, or a salt or solvate thereof, and wherein the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or a salt or solvate thereof.

Embodiment 9: The composition of any of Embodiments 1-8, wherein the composition causes pyroptosis in a cell of the melanoma.

Embodiment 10: A method of treating, preventing, and/or ameliorating melanoma in a subject in need thereof, the method comprising administering to the subject a MEK inhibitor; and at least one of a PDPK1 inhibitor and a PI3K inhibitor, or any salt or solvate thereof.

Embodiment 11: The method of Embodiment 10, wherein the melanoma is an NRAS mutant melanoma.

Embodiment 12: The method of any of Embodiments 10-11, wherein the subject is a mammal.

Embodiment 13: The method of any of Embodiments 10-12, wherein the subject is a human.

Embodiment 14: The method of any of Embodiments 10-13, wherein the MEK inhibitor comprises N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib) or N-[(2R)-2,3-dihydroxypropoxy]-3,4-difluoro-2-(2-fluoro-4-iodoanilino)benzamide (PD0325901), or any salt or solvate thereof.

Embodiment 15: The method of any of Embodiments 10-14, wherein the method comprises administering to the subject the PDPK1 inhibitor, and wherein the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or any salt or solvate thereof.

Embodiment 16: The method of any of Embodiments 10-15, wherein the method comprises administering the PI3K inhibitor, or any salt or solvate thereof, and wherein the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or any salt or solvate thereof.

Embodiment 17: The method of any of Embodiments 10-16, wherein the method causes pyroptosis in a cell of the melanoma.

Embodiment 18: A method of killing a melanoma cell, comprising: contacting the melanoma cell with a MEK inhibitor; and at least one of a PDPK1 inhibitor and a PI3K inhibitor.

Embodiment 19: The method of Embodiment 18, wherein the melanoma cell has a mutation in the NRAS gene.

Embodiment 20: The method of any one of Embodiments 18-19, wherein the melanoma cell is a cultured melanoma cell.

Embodiment 21: The method of Embodiment 20, wherein the melanoma cell is a cell of a melanoma cell line or a primary melanoma cell.

Embodiment 22: The method of any one of Embodiments 18-19, wherein the melanoma cell is in a subject.

Embodiment 23: The method of Embodiment 22, wherein the subject is a mammal.

Embodiment 24: The method of any one of Embodiment 22-23, wherein the subject is a human.

Embodiment 25: The method of any one of Embodiments 18-24, wherein the MEK inhibitor includes N-[3-[3-Cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide (Trametinib) or N-[(2R)-2,3-dihydroxypropoxy]-3,4-difluoro-2-(2-fluoro-4-iodoanilino)benzamide (PD0325901), or any salt or solvate thereof.

Embodiment 26: The method of any one of Embodiments 18-25, wherein the method comprises contacting the melanoma cell with the PDPK1 inhibitor, or any salt or solvate thereof, and wherein the PDPK1 inhibitor includes (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or any salt or solvate thereof.

Embodiment 27: The method of any one of Embodiments 18-26, wherein the method comprises contacting the melanoma cell with the PI3K inhibitor, and wherein the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or any salt or solvate thereof.

Embodiment 28: The method of any one of Embodiments 18-27, wherein the method causes pyroptosis in the melanoma cell.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A composition for treating, preventing, or ameliorating melanoma in a subject in need thereof, the composition comprising (a) MEK inhibitor, or any salt or solvate thereof, and (b) at least one of a PDPK1 inhibitor and a PI3K inhibitor, or any salt or solvate thereof.

2. The composition of claim 1, wherein at least one of the following applies:
(i) the melanoma is an NRAS mutant melanoma;
(ii) the composition causes pyroptosis in a cell of the melanoma;
(iii) the subject is a mammal; or
(iv) the subject is a human.

3-4. (canceled)

5. The composition of claim 1, further comprising a pharmaceutically acceptable excipient or carrier.

6. The composition of claim 1, wherein at least one of the following applies:
(i) the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or any salt or solvate thereof;
(ii) the composition comprises the PDPK1 inhibitor, or any salt or solvate thereof, and the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or any salt or solvate thereof;
(iii) the composition comprises the PI3K inhibitor, or any salt or solvate thereof, and the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or any salt or solvate thereof.

7-9. (canceled)

10. A method of treating, preventing, or ameliorating melanoma in a subject in need thereof, the method comprising administering to the subject (a) a MEK inhibitor, or any salt or solvate thereof, and (b) at least one of a PDPK1 inhibitor and a PI3K inhibitor, or any salt or solvate thereof.

11. The method of claim 10, wherein at least one of the following applies:
(i) the melanoma is an NRAS mutant melanoma;
(ii) the method causes pyroptosis in a cell of the melanoma;
(iii) the subject is a mammal; or
(iv) the subject is a human.

12-13. (canceled)

14. The method of claim 10, wherein at least one of the following applies:
(i) the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or any salt or solvate thereof;
(ii) the method comprises administering the PDPK1 inhibitor, or any salt or solvate thereof, and the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or any salt or solvate thereof; or
(iii) the method comprises administering the PI3K inhibitor, or any salt or solvate thereof, and the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or any salt or solvate thereof.

15-17. (canceled)

18. A method of killing a melanoma cell, the method comprising: contacting the melanoma cell with (a)la MEK inhibitor, or any salt or solvate thereof, and (b) at least one of a PDPK1 inhibitor and a PI3K inhibitor, or any salt or solvate thereof.

19. The method of claim 18, wherein at least one of the following applies:
(i) the melanoma cell has a mutation in the NRAS gene; or
(ii) the method causes pyroptosis in the melanoma cell.

20. The method of claim 18, wherein the melanoma cell is a cultured melanoma cell.

21. The method of claim 20, wherein the melanoma cell is a cell of a melanoma cell line or a primary melanoma cell.

22. The method of claim 18, wherein the melanoma cell is in a subject.

23. The method of claim 22, wherein at least one of the following applies:
(i) the subject is a mammal; or
(ii) the subject is a human.

24. (canceled)

25. The method of claim 18, wherein at least one of the following applies:
(i) the MEK inhibitor comprises at least one of 5-((4-bromo-2-fluorophenyl)amino)-4-fluoro-N-(2-hydroxyethoxy)-1-methyl-1H-benzo[d]imidazole-6-carboxamide (binimetinib), (S)-(3,4-difluoro-2-((2-fluoro-4-iodophenyl)amino)phenyl)(3-hydroxy-3-(piperidin-2-yl)azetidin-1-yl)methanone (cobimetinib), 5-((4-bromo-2-chlorophenyl)amino)-4-fluoro-N-(2-hydroxyethoxy)-1-methyl-1H-benzo[d]imidazole-6-carboxamide (selumetinib), N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), (S)—N-(2,3-dihydroxypropyl)-3-((2-fluoro-4-iodophenyl)amino)isonicotinamide (pimasertib), (R)—N-(2,3-dihydroxypropoxy)-3,4-difluoro-2-((2-fluoro-4-iodophenyl)amino)benzamide (PD-0325901), 2-((2-chloro-4-iodophenyl)amino)-3,4-difluorobenzoic acid (ATR-002 or zapnometinib), 5-((2-fluoro-4-iodophenyl)amino)-N-(2-hydroxyethoxy)imidazo[1,5-a]pyridine-6-carboxamide (GDC-0623), (S)—N-(3,4-difluoro-2-((2-fluoro-4-iodophenyl)amino)-6-methoxyphenyl)-1-(2,3-dihydroxypropyl)cyclopropane-1-sulfonamide (refametinib), (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK 733 or REC4881), and MSC2015103B (AS703988), or any salt or solvate thereof;
(ii) the method comprises contacting the melanoma cell with the PDPK1 inhibitor, or any salt or solvate thereof, and the PDPK1 inhibitor comprises (3S,6R)-1-[6-(3-Amino-1H-indazol-6-yl)-2-(methylamino)-4-pyrimidinyl]-N-cyclohexyl-6-methyl-3-piperidinecarboxamide (GSK2334470), or any salt or solvate thereof; or
(iii) the method comprises contacting the melanoma cell with the PI3K inhibitor, or any salt or solvate thereof, and wherein the PI3K inhibitor comprises at least one selected from the group consisting of acalisib, AEZS-136, alpelisib, AMG 319, AZD8186, AZD8835, apitolisib, B591, bimiralisib, buparlisib, CAL263, copanlisib, dactolisib, duvelisib, eganelisib, fimepinostat, gedatolisib, GNE-477, GSK1059615, GSK2636771, Hibiscone C, IC87114, idelalisib, inavolisib, leniolisib, linperlisib, LY294002, MEN1611, nemiralisib, omipalisib, parsaclisib, paxalisib, pictilisib, PI-103, pilaralisib, PWT33597, samotolisib, seletalisib, serabelisib, SF1126, sonolisib, taselisib, tenalisib, TG100-115, umbralisib, voxtalisib, Wortmannin, zandelisib, and ZSTK474, or any salt or solvate thereof.

26-28. (canceled)


SEQUENCE LISTING (9 sequences)

SEQ ID NO: 1 - DNA, 33 bp, synthetic construct
  source 1..33: mol_type = other DNA
ttggtcaccacatacgaagacgggtttaagagc

SEQ ID NO: 2 - DNA, 40 bp, synthetic construct
  source 1..40: mol_type = other DNA
ttagctcttaaacccgtcttcgtatgtggtgaccaacaag

SEQ ID NO: 3 - RNA, 40 nt, synthetic construct
  source 1..40: mol_type = other RNA
ttagctcttaaacccgtcttcgtatgtggtgaccaacaag

SEQ ID NO: 4 - RNA, 21 nt, synthetic construct
  source 1..21: mol_type = other RNA
caagagacctcgtggagaatt

SEQ ID NO: 5 - RNA, 19 nt, synthetic construct
  source 1..19: mol_type = other RNA
gaccagaggccaagaattt

SEQ ID NO: 6 - DNA, 25 bp, synthetic construct
  source 1..25: mol_type = other DNA
caccgcaagtttgggaaaatccttg

SEQ ID NO: 7 - DNA, 25 bp, synthetic construct
  source 1..25: mol_type = other DNA
aaaccaaggattttcccaaacttgc

SEQ ID NO: 8 - DNA, 25 bp, synthetic construct
  source 1..25: mol_type = other DNA
caccgcccgctctctggttacatag

SEQ ID NO: 9 - DNA, 25 bp, synthetic construct
  source 1..25: mol_type = other DNA
aaacctatgtaaccagagagcgggc